(19) United States
(12) Patent Application Publication
(10) Pub. No.: US 2020/0147215 A1
(43) Pub. Date: May 14, 2020

(54) DRUG CONJUGATES WITH PHOTOCLEAVABLE SOLUBILITY MODULATORS

(71) Applicants: SIMON H. FRIEDMAN, KANSAS CITY, MO (US); KARTHIK NADENDLA, KANSAS CITY, MO (US); BHAGYESH SARODE, KANSAS CITY, MO (US); PIYUSH JAIN, KANSAS CITY, MO (US); DIPU KARUNAKARAN, KANSAS CITY, MO (US); SWETHA CHINTALA, KANSAS CITY, MO (US); PARTH SHAH, KANSAS CITY, MO (US)

(72) Inventors: SIMON H. FRIEDMAN, KANSAS CITY, MO (US); KARTHIK NADENDLA, KANSAS CITY, MO (US); BHAGYESH SARODE, KANSAS CITY, MO (US); PIYUSH JAIN, KANSAS CITY, MO (US); DIPU KARUNAKARAN, KANSAS CITY, MO (US); SWETHA CHINTALA, KANSAS CITY, MO (US); PARTH SHAH, KANSAS CITY, MO (US)

(21) Appl. No.: 16/607,573

(22) PCT Filed: Apr. 24, 2018
(86) PCT No.: PCT/US2018/029056

§ 371 Date: Oct. 23, 2019

Related U.S. Application Data
(60) Provisional application No. 62/489,163, filed on Apr. 24, 2017.

(51) Int. Cl. A61K 41/00; A61K 47/64; A61K 47/54; A61K 9/00; A61N 5/06

(52) U.S. Cl. CPC A61K 41/0042; A61K 47/64; A61K 47/54; A61N 2005/0626; A61N 5/062; A61N 2005/0651; A61K 9/0024

(57) ABSTRACT

The present invention is directed to a composition suitable for forming an implanted light activated drug depot, methods of making the composition, and methods and systems for using the composition. The composition comprises a plurality of drug conjugates, which comprise a drug molecule and a small solubility modulating portion. The drug conjugates are insoluble upon implantation as a drug depot into a subject, and the drug is preferably soluble once cleaved from the depot. One aspect of the invention is directed to a drug conjugate having a modulating portion that modifies the solubility of the drug conjugate by employing a hydrophobic non-polar moiety. Another aspect of the invention is directed to a drug conjugate having a modulating portion that modifies the solubility of the drug by employing a charged moiety that shifts the isoelectric point of the drug conjugate to a physiological pH.

### CROSS-REFERENCE TO RELATED APPLICATIONS

This application is based on and claims priority to U.S. Provisional Application Ser. No. 62/489,163 filed on Apr. 24, 2017, which is hereby incorporated herein by reference.

### BACKGROUND OF THE INVENTION

There are many drugs, especially protein based drugs, that would benefit from controlled release in response to physiological signals. A prime example of this is insulin, as used by diabetics, which needs to be administered multiple times per day, in varying amounts, in response to changing blood sugar levels.

Materials that comprise insulin linked to a polymer with a light-cleaved linker have been previously described. An example of such polymer-based drug conjugate is depicted in FIG. 1. The purpose of the polymer is to make the material insoluble so that when particles of it are injected into the skin, they remain there, and can then be irradiated with a light source. In a diabetic animal, insulin can be released from such materials after irradiation with a light source, and blood sugar is subsequently reduced.

Such polymer-based drug conjugates have limitations. For example, a large amount of the material consists of polymer, making the materials low density in insulin. This has two problems associated with it. The materials require more light to release, and have a shorter duration of action because of lower amounts of insulin. In addition, the materials leave behind the polymer after photolysis, which requires some mechanism to clear from the body, such as physical removal or biodegradation. Both of these methods create significant practical problems.

### BRIEF SUMMARY OF THE INVENTION

The present invention is directed to novel drug conjugates having a small photocleavable solubility modulating portion, and drug delivery methods and systems which use such conjugates.

One aspect of the present invention is directed to a composition for forming an implanted drug depot that comprises a plurality of drug conjugates. The drug conjugates include (a) a solubility modulating portion that comprises a biocompatible, bioresorbable moiety and a photocleavable group linked to said moiety, and (b) a drug molecule linked to the photocleavable group of the modulating portion. The drug conjugates are insoluble at physiological pH.

In one aspect of the invention, the modulating portion, including the moiety, are small. In certain aspects both the modulating portion and moiety have a molecular weight of 2000 or less, preferably 1500 or less, more preferably 1000 or less.

In one aspect of the invention, the moiety is soluble at physiological pH. In some such embodiments, the moiety is non-polar.

In one aspect of the invention, the moiety may be a peptide comprising 20 or fewer non-polar amino acids, preferably 15 or fewer, 10 or fewer, or 5 or fewer non-polar amino acids. In some such embodiments, the moiety comprises 3 non-polar amino acids.

In one aspect of the invention, the moiety is comprised of amino acids selected from the group consisting of glycine, alanine, valine, leucine, isoleucine, proline, phenylalanine, methionine, tyrosine and tryptophan. In some such embodiments, the moiety comprises a valine-proline-isoleucine peptide or a valine-valine-valine peptide.

In another aspect of the invention, the moiety is a substituted or unsubstituted hydrocarbon. In one such aspect, the moiety comprises cyclododecyl amine.

In yet another aspect of the invention, the moiety has a charge that shifts the isoelectric point of the drug conjugate to a physiological pH. In some such aspects, the physiological pH is from 6.5 to 7.5.

In another aspect of the invention, the moiety comprises one or more groups selected from positive groups, negative groups and combinations thereof, wherein the combined charge of said moiety shifts the isoelectric point of the drug conjugate to a physiological pH. In some such aspects, the drug molecule is insulin and said moiety adds two positive charges to the drug conjugate.

In another aspect of the invention, the moiety is charged and the moiety comprises a peptide. In some such aspects, the peptide comprises amino acids selected from the group consisting of arginine, lysine and histidine. In some such embodiments, the peptide comprises two amino acids. In some such embodiments, the peptide is an arginine-arginine peptide.

In one aspect of the invention, the moiety comprises glutamic acid that has been condensed with two 1-(2-Aminoethyl)pyrrolidine moieties (G2PEA).

In one aspect of the invention, the drug is a therapeutic peptide. In some such embodiments, the therapeutic peptide is insulin.

Another aspect of the invention is directed to method of administering a drug to a patient that comprises implanting the composition of any of the aspects of the invention into a patient to form said depot, and transdermally irradiating said implanted depot with light sufficient to cleave said photocleavable group and release said drug molecule from the drug conjugate, wherein said released drug molecule is in its native form. In some such embodiments, the implanting step comprises injecting said depot cutaneously or subcutaneously.

Another aspect of the invention is directed to a system for administering a drug to a patient that comprises the composition comprising a drug conjugate according to any of the aspects of the present invention and a light emitting device. In some such embodiments, the light emitting device is in the form of a band, patch, or bandage adapted to be positioned on said patient's skin. In some such embodiments, the light emitting device is programmed to provide light in response to a biological variable in a patient and wherein said system further comprises a sensor for measuring said biological variable to provide feedback to said light emitting device.

Additional aspects of the invention, together with the advantages and novel features appurtenant thereto, will be set forth in part in the description which follows, and in part will become apparent to those skilled in the art upon examination of the following, or may be learned from the practice of the invention. The objects and advantages of the invention may be realized and attained by means of the instrumentalities and combinations particularly pointed out in the appended claims.

### BRIEF DESCRIPTION OF THE DRAWINGS

FIG. 1 depicts a prior art polymer-drug conjugate.

FIG. 2 depicts the components of a drug conjugate of the present invention comprising a non-polar moiety.

FIG. 3 depicts the components of a drug conjugate of the present invention comprising a charged moiety.

FIG. 4 depicts an exemplary synthesis scheme for a drug conjugate of the present invention comprising insulin and a cyclododecyl amine moiety.

FIG. 5 shows the mass spectrometry (MS) characterization of a drug conjugate of the present invention comprising insulin and cyclododecyl amine moiety.

FIG. 6 shows the solubility of insulin and a drug conjugate of the present invention comprising insulin and cyclododecyl amine moiety.

FIG. 7 shows the photolysis release profile of a drug conjugate of the present invention comprising insulin and cyclododecyl amine moiety.

FIG. 8 depicts an exemplary synthesis scheme for a drug conjugate of the present invention comprising insulin and a valine-proline-isoleucine moiety.

FIG. 9 depicts a schematic of a portion of an exemplary synthesis scheme.

FIG. 10A shows the MS characterization of the ketone intermediate of a drug conjugate of the present invention comprising insulin and a valine-proline-isoleucine moiety.

FIG. 10B depicts the structure of certain compounds detected by the MS shown in FIG. 10A.

FIG. 11 shows the MS characterization of the hydrazone intermediate of a drug conjugate of the present invention comprising insulin and a valine-proline-isoleucine moiety FIG. 12 shows the MS confirmation of synthesis of valine-proline-isoleucine-hydrazone.

FIG. 13 depicts the fragments detected by the MS shown in FIG. 12

FIG. 14A shows the confirmation of reaction of a hydrazone with model compound PBA.

FIG. 14B depicts the structure of the molecule.

FIG. 15 depicts reactions competing with the diazotization reaction.

FIG. 16 shows the confirmation of an azine formed from stored hydrazone.

FIG. 17A shows HPLC confirmation of formation of a drug conjugate of the present invention comprising insulin and a valine-proline-isoleucine moiety.

FIG. 17B shows MS confirmation of formation of a drug conjugate of the present invention comprising insulin and a valine-proline-isoleucine moiety.

FIG. 17C depicts the structure of drug conjugate.

FIG. 18 shows the solubility of insulin and a drug conjugate of the present invention comprising insulin and a valine-proline-isoleucine moiety.

FIG. 19 shows the photolysis release profile of a drug conjugate of the present invention comprising insulin and a valine-proline-isoleucine moiety.

FIG. 20 depicts an exemplary synthesis scheme for valine-valine-valine-NKA.

FIG. 21A shows the liquid chromatography-mass spectrometry (LCMS) characterization of valine-valine-valine-NKA.

FIG. 21B depicts the structure of valine-valine-valine-NKA.

FIG. 22A shows the LCMS characterization of a valine-valine-valine-hydrazone.

FIG. 22B depicts the structure of valine-valine-valine-hydrazone.

FIG. 23 depicts the last steps in an exemplary synthesis scheme for a drug conjugate of the present invention comprising insulin and a valine-valine-valine moiety.

FIG. 24A shows the LCMS characterization of a drug conjugate of the present invention comprising insulin and a valine-valine-valine moiety.

FIG. 24B depicts the structure of the drug conjugate.

FIG. 25 shows the photolysis release profile of a drug conjugate of the present invention comprising insulin and a valine-valine-valine moiety.

FIG. 26 depicts an exemplary synthesis scheme for a drug conjugate of the present invention comprising insulin and an arginine-arginine moiety.

FIG. 27A shows the MS characterization of an insulin fraction.

FIG. 27B shows the MS characterization of a drug conjugate of the present invention comprising insulin and an arginine-arginine amine moiety.

FIG. 28 depicts a drug conjugate of the present invention comprising insulin and a G2PEA moiety.

FIG. 29 depicts the steps of an exemplary synthesis scheme for a drug conjugate of the present invention comprising insulin and a G2PEA moiety.

FIG. 30 shows the LCMS characterization of G2PEA-hydrazone.

FIG. 31 shows the LCMS characterization of G2PEA-NKA.

FIG. 32 shows the MS characterization of a drug conjugate of the present invention comprising insulin and a G2PEA moiety.

FIG. 33 shows the altered isoelectric point of G2PEA using an IEF gel.

FIG. 34 shows the solubility of G2PEA at pH 4 and pH 7.

FIG. 35 shows the photolysis release profile of a drug conjugate of the present invention comprising insulin and a G2PEA over time analyzed by a gel.

FIG. 36 shows the photolysis release profile of a drug conjugate of the present invention comprising insulin and a G2PEA moiety in DMSO.

FIG. 37 shows the photolysis release profile of a drug conjugate of the present invention comprising insulin and a G2PEA moiety in PBS at pH 7.2.

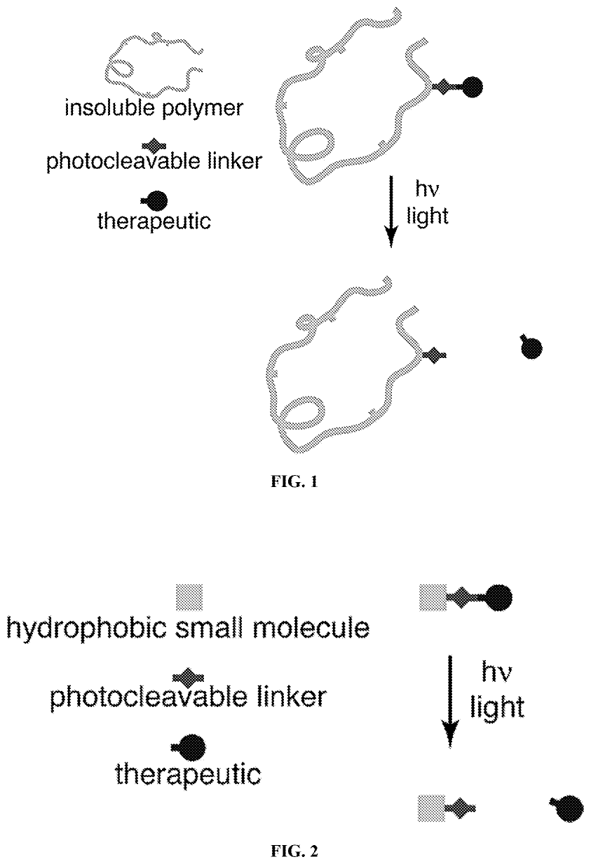

### DETAILED DESCRIPTION OF PREFERRED EMBODIMENT

For the purposes of this specification and appended claims, unless otherwise indicated, all numbers expressing quantities of ingredients, percentages or proportions of materials, reaction conditions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth, the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “1 to 10” includes any and all subranges between (and including) the minimum value of 1 and the maximum value of 10, that is, any and all subranges having a minimum value of equal to or greater than 1 and a maximum value of equal to or less than 10, e.g., 5.5 to 10.

It is noted that, as used in this specification and the appended claims, the singular forms “a,” “an,” and “the” include plural referents unless expressly and unequivocally limited to one referent. Thus, for example, reference to “a molecule” includes molecules.

Reference will now be made in detail to certain embodiments of the invention, examples of which are illustrated in the accompanying drawings. While the invention will be described in conjunction with the illustrated embodiments, it will be understood that they are not intended to limit the invention to those embodiments. On the contrary, the invention is intended to cover all alternatives, modifications, and equivalents, which may be included within the invention as defined by the appended claims.

The present invention is directed to novel compositions of matter and methods for drug delivery. In particular, the present invention is generally directed to a composition that permits the toggling of the release of drugs inside the body by using an implantable, preferably injectable, light activated drug depot. Although insulin will be used to describe the composition and delivery approach, it will be readily appreciated that the present invention can be applied to any molecule in which controlled and/or timed release is desired to maximize effectiveness. Such molecules include but are not limited to small molecule drugs, peptides, proteins, nucleic acids, and macromolecules.

In one aspect, the present invention is directed to a composition suitable for forming an implanted light activated drug depot. The composition comprises a plurality of drug conjugates. The drug conjugates comprise a drug molecule and a solubility modulating portion. The drug conjugates are insoluble upon implantation as a drug depot into a subject.

The term “insoluble” when applied to the drug conjugate means the drug conjugate is insoluble in an aqueous medium. When used herein “insoluble” encompasses very slightly soluble in the solute (requiring 1000 to 10,000 mass parts of solvent to dissolve 1 mass part of solute) and practically insoluble (requiring 10,000 or greater mass parts of solvent to dissolve 1 mass part of solute). Further, the drug conjugate is insoluble at the physiological pH existing upon implantation into a subject. Most drug conjugates will be injected into a physiological pH that is around 7, for example, greater than 6, greater than 6.1, 6.2, 6.3, 6.4, 6.5, 6.6, 6.7, 6.8 or 6.9 and less than 8, or less than 7.9, 7.8, 7.7, 7.6, 7.5. 7.4, 7.3, 7.2, or 7.1 and any ranges therebetween.

The solubility modulating portion of the drug conjugate includes a biocompatible, bioresorbable moiety, which modulates the solubility of the drug conjugate, and a photocleavable group (PC) linking the drug molecule to the solubility modulating portion. Upon exposure to light of a suitable wavelength, the solubility modulating portion is cleaved from the drug molecule. The cleaved drug molecule is preferably soluble in an aqueous medium and at a physiological pH. The depot comprising the drug conjugate of the present invention allows for controlled release from the light-activated depot. The released drug molecule is preferably in its native form without additions.

The drug conjugate can generally be described as:

Moiety-PC-Drug.

In general, the photocleavable group and moiety may be thought of as part of the solubility modulating portion. In certain embodiments, the photocleavable group significantly contributes to the insolubility of the conjugate. However, in most embodiments, the photocleavable group will not itself materially modulate the solubility of the drug conjugate.

The present invention provides a drug conjugate that forms a drug depot in which the drug molecule is highly concentrated. Because of this high concentration it has the potential to release the drug molecule easily, with low amounts of light. Also, it has the potential to reduce the overall volume of injected material, reducing the discomfort associated with injection. In addition, a given volume of the drug depot can contain many doses, extending the duration for which the depot can act. Finally, the modulating portion released from the drug molecule upon photolysis is small (smaller than a polymer) and will much more easily be cleared from the system. All of this is in contrast to a polymer linked to a drug molecule, which forms a depot with low drug density and shorter duration of use, requires more light, and is too large to be absorbed by itself and must have the ability to be biodegraded. The combined features of the present invention increase the utility of the present invention over polymer-based drug conjugates.

Certain elements of the invention will now be described in more detail.

Moiety of the Modulating Portion

The modulating portion, and the moiety comprising the modulating portion, are small, which provides the benefits discussed above over polymer-based drug conjugates. The complete modulating portion is only slightly larger than the moiety, due to the addition of a small photocleavable group. Preferably the modulating portion, and necessarily the moiety that is part of the modulating portion, has a molecular weight of 2000 Da or less, preferably 1500 Da or less, more preferably 1000 Da or less, or 1900, 1800, 1700, 1600, 1500, 1400, 1300, 1200, 1100, 1000, 900, 800, 700, 600 500 Da or less, and all values and ranges therebetween.

The small size of the moiety allows for the depot to comprise primarily pharmaceutical ingredients, which allows for a high drug loading. Preferably the modulating portion, and necessarily the moiety comprising the modulation portion, makes up less than 50% of the depot, more preferably less than 15%, 10% or 5% of the depot by weight. In some aspects, the modulating portion, and necessarily the moiety comprising the modulation portion, make up about 90%, 89%, 88%, 87%, 86%, 85%, 84%, 83%, 82%, 81%, 80%, 79%, 78%, 77%, 76%, 75%, 74%, 73%, 72%, 71%, 70%, 65%, 60%, 55%, 50%, 45%, 40%, 35%, 30%, 25%, 20%, 19%, 18%, 17%, 16% 15%, 14%, 13%, 12%, 11%, 10%, 9%, 8%, 7%, 6%, 5%, 4%, 3%, 2%, 1%, or any value or range therebetween, of the total weight of the depot, and the remainder is active and/or inactive pharmaceutical ingredients.

Because the moieties are small molecules, there is no need for further biodegradation of the modulating portion after the depot releases its drug cargo. The moieties can be easily cleared from the body after photolysis by natural pathways. This is a benefit over long polymer chains that must be biodegraded prior to uptake by the body. The modulating portion of the drug conjugate of the present invention does not comprise a long polymer chain and is not a polymer backbone to which multiple drugs are crosslinked.

In one aspect, the moiety is preferably bioresorbable. As used here, the term bioresorbable refers to a moiety whose degradative products, or the moiety itself, are metabolized in vivo or excreted from the body via natural pathways. In general, by “bioresorbable,” it is meant that the depot will be broken down and absorbed within the human body, for example, by a cell or tissue.

In one aspect, the moiety is preferably biocompatible. As used herein the term “biocompatible” means that the moiety (and thus the modulating portion and depot) will not cause substantial tissue irritation or necrosis at the target tissue site. Preferably, the moiety is approved for use in the body by the Food and Drug Administration.

As noted above, most drug molecules are soluble at a physiological pH. The present invention modulates the solubility of the drug molecule to achieve low solubility of the drug conjugate prior to light irradiation and normal solubility afterward. This allows the insoluble drug conjugates to be implanted as a drug depot that will stay at the location of implantation. Release of the drug molecules from the depot can be controlled through controlled light irradiation.

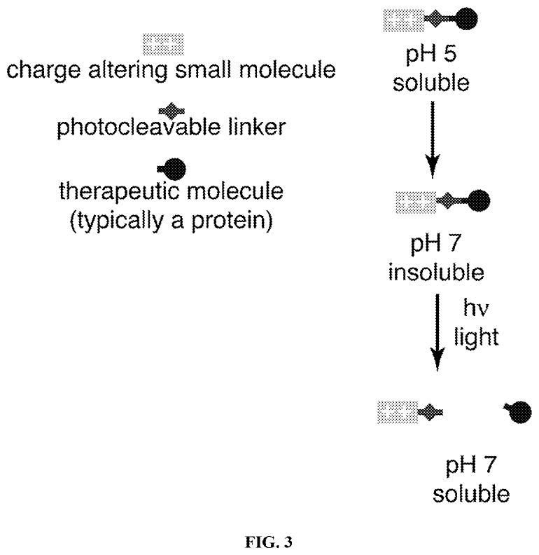

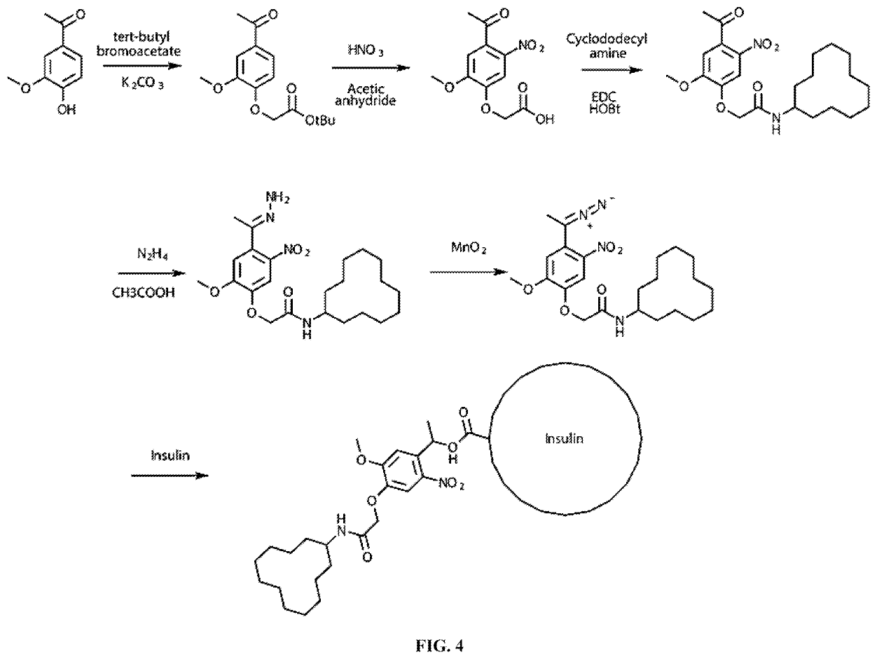

One aspect of the invention is directed to drug conjugates having a modulating portion that modifies the solubility of the drug conjugate by employing a hydrophobic non-polar moiety. FIG. 2 is an exemplary illustration of such aspect. Another aspect of the invention is directed to a drug conjugate having a modulating portion that modifies the solubility of the drug by employing a charged moiety that shifts the isoelectric point of the drug conjugate to a physiological pH. FIG. 3 is an exemplary illustration of such aspect. Both of these aspects are discussed in more detail below.

### Insoluble Moiety

In one aspect of the invention, the drug conjugate is rendered insoluble by a highly non-polar hydrophobic moiety. “Non-polar” can be defined has having an octanol/water partition coefficient above 0. Thus, rather than linking the drug molecule to a large insoluble polymer as has been done in the prior art, the present invention uses small non-polar moieties to render the drug conjugate insoluble. Because of their highly non-polar nature, when linked to a drug molecule such as insulin, the non-polar moiety makes the drug conjugate insoluble. The drug conjugate can thus form small injectable but insoluble particles that can be implanted to form the depot. When the drug molecules, such as insulin, are needed, the depot material can be irradiated, which cleaves the non-polar moiety from the drug molecule, causing drug molecule's solubility to increase, and for it to be released from the depot. The cleaved drug is preferably in its native form, without additions.

The non-polar moiety can be naturally based, for example a peptide, or non-natural, such as a cyclododecyl amine. As demonstrated in Examples 1-3, the drug conjugates comprising a non-polar moiety have much less solubility than the drug molecule alone.

In certain aspects of the invention, the moiety is a non-polar peptide comprising 20 or fewer non-polar amino acids, preferably 10 or fewer non-polar amino acids. The non-polar peptide may comprise 20, 19, 18, 17, 16, 15, 14, 13, 12, 11, 10, 9, 8, 7, 6, 5, 4, 3, 2 or even 1 amino acids. The amino acids are preferably non-polar amino acids selected from the group consisting of glycine, alanine, valine, leucine, isoleucine, proline, phenylalanine, methionine, tyrosine and tryptophan. Although it is preferred that all amino acids are non-polar, to maximize the effect of the moiety and minimize the size, it is contemplated that a combination of polar and non-polar amino acids that produce an overall non-polar moiety could be used.

In the exemplary embodiments shown in the examples, the moiety comprises 3 non-polar amino acids. In certain exemplary embodiments, the moiety comprises a valine-proline-isoleucine peptide or a valine-valine-valine peptide.

In addition to peptides, any other sufficiently small non-polar groups may be employed in the non-polar moieties of the present invention. For example, substituted or unsubstituted hydrocarbons may be used consistent with the present invention. Exemplary non-polar groups for that may be included in the non-polar moiety include fatty acids, steroids, fatty alcohols, derivatives of alkanes, alkenes and alkynes, and derivatives of aryl groups. In certain exemplary embodiments, the moiety comprises a cycloalkane. In one exemplary embodiment shown in the example, the moiety is a cyclododecyl amine.

### Charged Moiety

In one aspect of the invention, the drug conjugate is rendered insoluble by a moiety that shifts the iso-electric point (pI) of the drug conjugate to a physiological pH. For example, insulin's isoelectric point is ˜5.4. This means it is highly soluble at the neutral pH of the body (˜7) but has very low solubility at 5.4. This is because at a pH of 5.4 (the isoelectric point), the overall protein has 0 net charge. All of the negative and positive groups exactly cancel out. When a protein has no net charge, it has its lowest solubility.

In the present invention the moiety has a charge that shifts the isoelectric point (pI) of the drug conjugate to a physiological pH. In the case of insulin, the moiety of the modulating portion adds positive charges. This shifts the pI of the insulin to be ˜7, the pH of the body. As a result, the drug conjugate comprising insulin can be formulated at a low pH, away from the new pI, a pH at which it is highly soluble. It can then be easily injected as it is a completely homogenous solution. Once it enters the body where the pH is ˜7 (the pI of the drug conjugate), the drug conjugate immediately precipitates. This is because in such pH environment the drug conjugate has no net charge, and has its lowest solubility. The insolubility results from the match between the pH of the physiological fluid and the new pI of the drug conjugate comprising the insulin. The insoluble drug conjugate can form a drug depot at the location it is implanted, such as the skin.

When the drug, such as insulin, is needed to be released from the depot, it is irradiated with light. The photocleavable group breaks its bond with the drug molecule, removing the charged groups from the drug molecule. The drug molecule is then in its native form with no additions. In the case of insulin, after cleavage from the drug conjugate, its pI is 5.4, meaning that in the body at ˜7 it is highly soluble. Light has triggered the release of insulin and it can now be absorbed into the body via vasculature away from the depot site.

As discussed above, the desired physiological pH will be the pH of the location of the body into which the drug conjugates are implanted, such as the skin. Most drug conjugates will be injected into a physiological pH that is around 7. A physiological pH may be, for example, greater than 6, greater than 6.1, 6.2, 6.3, 6.4, 6.5, 6.6, 6.7, 6.8 or 6.9 and less than 8, or less than 8.9, 8.8, 8.7, 8.6, 8.5. 8.4, 8.3, 8.2, or 8.1 and any ranges therebetween. In certain embodiments, the physiological pH is from 6.5 to 7.5.

The moiety may comprise positive groups, negative groups and combinations thereof, wherein the combined charge of said moiety shifts the isoelectric point of the drug conjugate to a physiological pH. It is contemplated that a combination of positive and negative charged groups that produce an overall charged moiety could be used. Further, in certain embodiments, attachment of the photocleavable group can affect the charge of the drug molecule. For example, the attachment point of DMNPE is a carboxyl group on the surface of the insulin. The modification of this carboxyl removes one negative charge (as the carboxyl can develop a negative charge at pH 7) and thus has the effect of adding an additional positive charge.

The total charge of the moiety will depend on the drug molecule. For example, when the drug molecule is insulin, the moiety adds two positive charges to the drug conjugate.

The charged moiety can be naturally based, for example a peptide, or non-natural. As demonstrated in Examples 4-5, the drug conjugates comprising a charged moiety have much less solubility than the drug molecule alone.

The moiety may comprise a peptide. The peptide preferably comprises amino acids selected from the group consisting of arginine, lysine and histidine. In certain embodiments, the moiety comprises two amino acids, which may be the same or different. In one exemplary embodiment shown in the examples, the peptide is an arginine-arginine peptide.

In addition to peptides, any group that is sufficiently small and comprises the total charge to counter the charge of the drug molecule and shift the isoelectric point to the physiological pH may be used. In some case, a group with a single net charge may be sufficient. In one exemplary embodiment shown in the examples, the moiety comprises glutamic acid that has been condensed with two 1-(2-Aminoethyl)pyrrolidine moieties (G2PEA).

### Characteristics Applicable to Insoluble and Charged Moieties

The moiety may have a synthetic “handle,” i.e., a reactive group or functionality that will allow it to be joined to a photocleavable group. It will be appreciated that there are a wide variety of possible functionalities that are possible in this regard. Exemplary reactive groups include, but are not limited to hydroxyl, amine, carboxyl, (such as carboxylic acid, amide, carboxylic halide, carboxylic acid ester or carboxylic acid anhydride, and the carboxyl group may be activated, as is well known in the art, to facilitate coupling), vinylsulfone, alkyne, azide, maleimide, isothiocyanate, isocyanate, imidate, alpha-halo-amide, Michael acceptor, hydrazide, oxyamine, thiol, hydrazine, or a combination thereof. The embodiments wherein the moiety comprises multiple groups in a chain (e.g. a tripeptide or oligomer), the handle may be a side chain extending from the primary chain and/or at the terminal end of the chain.

In one aspect, the moiety has a carboxylic acid functionality. In such a case, the moiety can be linked to the photocleavable group via an amine on the photocleavable group. That is, the moiety is linked to the photocleavable linker via an amide bond.

In one aspect, the moiety has an amine functionality. In such a case, the moiety can be linked to the photocleavable group via a carboxylic acid on the photocleavable group. That is, the moiety is linked to the photocleavable linker via an amide bond.

In another aspect, the moiety has an azide functionality. In such a case, the moiety can be linked to the photocleavable group via an alkyne on the photocleavable group. That is, the moiety is linked to the photocleavable linker via a triazole bridge.

In still another aspect, the moiety has an alkyne functionality. In such a case, the moiety can be linked to the photocleavable group via an azide on the photocleavable group. That is, the moiety is linked to the photocleavable linker via a triazole bridge.

Photocleavable Group

The drug conjugate of the present invention comprises a photocleavable group. The photocleavable group links the solubility modulating portion of the drug conjugate to the drug molecule.

In one aspect, the photocleavable groups have at least two synthetic “handles” or reactive groups. The first reactive group allows linking of the photocleavable group to the moiety. The second reactive group allows linking of the photocleavable group to the drug molecule (such as insulin). The former handle is preferably stable and the latter handle is preferably amenable to photolysis such that the drug (e.g., insulin) cargo is released from the photocleavable drug conjugate upon exposure to light of the appropriate wavelength. The photocleavable group may be a bifunctional or multifunctional photocleavable group that could bind multiple drug molecules and/or multiple moieties.

In one aspect, the photocleavable group has a carboxylic acid functionality. In such a case, the moiety may be linked to the photocleavable group via an amine on the moiety. That is, the moiety may be linked to the photocleavable linker via an amide bond.

In one aspect, the photocleavable group has an amine functionality. In such a case, the moiety may be linked to the photocleavable group via a carboxylic acid on the moiety. That is, the moiety may be linked to the photocleavable linker via an amide bond.

In another aspect, the photocleavable group has an azide functionality. In such a case, the moiety may be linked to the photocleavable group via an alkyne on the moiety. That is, the moiety may be linked to the photocleavable linker via a triazole bridge.

In still another aspect, the photocleavable group has an alkyne functionality. In such a case, the moiety may be linked to the photocleavable group via an azide on the polymer. That is, the moiety may be linked to the photocleavable linker via a triazole bridge.

In another aspect, the photocleavable group has a diazo functionality. In such a case, the drug molecule (such as insulin) may be linked to the photocleavable group via a carboxylic acid functional group on the drug molecule. That is, the drug molecule may be linked to the photocleavable linker via an ester bond.

In another aspect, the photocleavable group has an N-hydroxy succinamide (“NHS”) ester functionality. The drug molecule may be linked to the photocleavable group via an amine on the drug molecule. That is, the drug molecule may be linked to the photocleavable linker via a carbamate/urethane bond.

In another aspect, the photocleavable group has an imidazole functionality.

The drug molecule may be linked to the photocleavable group via an amine on the drug molecule. That is, the drug molecule may be linked to the photocleavable linker via a carbamate bond.

In general, the photocleavable group should also have minimal toxicity. The photochemical properties of the photocleavable groups include any agent which may be linked to the drug molecule and which, upon exposure to light, releases the drug in functional form (or a suitable prodrug form). In general, groups capable of longer wavelength photolysis will show more efficient cleavage at deeper levels.

Exemplary photocleavable groups are generally described and reviewed in Pelliccioli et al., Photoremovable protecting groups: reaction mechanisms and applications, Photochem. Photobiol. Sci. 1 441-458 (2002); Goeldner and Givens, Dynamic Studies in Biology, Wiley-VCH, Weinheim (2005); Marriott, Methods in Enzymology, Vol. 291, Academic Press, San Diego (1998); Morrison, Bioorganic Photochemistry, Vol. 2, Wiley, New York (1993); Adams and Tsien, Annu. Rev. Physiol. 55 755-784 (1993); Mayer et al., Biologically Active Molecules with a “Light Switch,” Angew. Chem. Int. Ed. 45 4900-4921 (2006); Pettit et al., Neuron 19 465-471 (1997); Furuta et al., Brominated 7-hydroxycoumarin-4-ylmethyls: Photolabile protecting groups with biologically useful cross-sections for two photon photolysis, Proc. Natl. Acad. Sci. USA 96 1193-1200 (1999); and U.S. Pat. Nos. 5,430,175; 5,635,608; 5,872,243; 5,888,829; 6,043,065; and Zebala, U.S. Patent Application No. 2010/0105120, which are incorporated by reference herein with respect to such disclosures.

The photocleavable group may generally be described as a chromophore.

Examples of chromophores which are photoresponsive to such wavelengths include, but are not limited to, acridines, nitroaromatics, coumarins and arylsulfonamides. The efficiency and wavelength at which the chromophore becomes photoactivated and thus releases the drug will vary depending on the particular functional group(s) attached to the chromophore. For example, when using nitroaromatics, such as derivatives of o-nitrobenzylic compounds, the absorption wavelength can be significantly lengthened by addition of methoxy groups.

In one aspect, the photocleavable group is a nitro-aromatic compound.

Exemplary photocleavable groups having an ortho-nitro aromatic core scaffold include, but are not limited to, ortho-nitro benzyl (“ONB”), 1-(2-nitrophenyl)ethyl (“NPE”), alpha-carboxy-2-nitrobenzyl (“CNB”), 4,5-dimethoxy-2-nitrobenzyl (“DMNB”), 1-(4,5-dimethoxy-2-nitrophenyl)ethyl (“DMNPE”), 5-carboxymethoxy-2-nitrobenzyl (“CMNB”) and ((5-carboxymethoxy-2-nitrobenzyl)oxy)carbonyl (“CMNCBZ”) photolabile cores. It will be appreciated that the substituents on the aromatic core are selected to tailor the wavelength of absorption, with electron donating groups (e.g., methoxy) generally leading to longer wavelength absorption. For example, nitrobenzyl (“NB”) and nitrophenylethyl (“NPE”) are modified by addition of two methoxy residues into 4,5-dimethoxy-2-nitrobenzyl and 1-(4,5-dimethoxy-2-nitrophenyl)ethyl, respectively, thereby increasing the absorption wavelength range to 340-360 nm.

Further, other ortho-nitro aromatic core scaffolds include those that trap nitroso byproducts in a hetero Diels Alder reaction as generally discussed in Zebala, U.S. Patent Application No. 2010/0105120 and Pirrung et al., J. Org. Chem. 68:1138 (2003). The nitrodibenzofurane (“NDBF”) chromophore offers an extinction coefficient significantly higher in the near UV region but it also has a very high quantum yield for the deprotection reaction and it is suitable for two-photon activation (Momotake et al., The nitrodibenzofuran chromophore: a new caging group for ultra-efficient photolysis in living cells, Nat. Methods 3 35-40 (2006)). The NPP group is an alternative introduced by Pfleiderer et al. that yields a less harmful nitrostyryl species (Walbert et al., Photolabile Protecting Groups for Nucleosides: Mechanistic Studies of the 2-(2-Nitrophenyl)ethyl Group, Helv. Chim. Acta 84 1601-1611 (2001)).

In an exemplary aspect involving UV light, the photocleavable group is selected from the group consisting of alpha-carboxy-2-nitrobenzyl (CNB, 260 nm), 1-(2-nitrophenyl)ethyl (NPE, 260 nm), 4,5-dimethoxy-2-nitrobenzyl (DMNB, 355 nm), 1-(4,5-dimethoxy-2-nitrophenyl)ethyl (DMNPE, 355 nm), (4,5-dimethoxy-2-nitrobenzoxy)carbonyl (NVOC, 355 nm), 5-carboxymethoxy-2-nitrobenzyl (CMNB, 320 nm), ((5-carboxymethoxy-2-nitrobenzyl)oxy)carbonyl (CMNCBZ, 320 nm), desoxybenzoinyl (desyl, 360 nm), and anthraquino-2-ylmethoxycarbonyl (AQMOC, 350 nm).

Other suitable photocleavable groups are based on the coumarin system, such as BHC (Furuta and Iwamura, Methods Enzymol. 291 50-63 (1998); Furuta et al., Proc. Natl. Acad. Sci. USA 96 1193-1200 (1999); Suzuki et al., Org. Lett. 5:4867 (2003); U.S. Pat. No. 6,472,541, which are incorporated by reference herein with respect to such disclosures. The DMACM linkage photocleaves in nanoseconds (Hagen et al., [7-(Dialkylamino)coumarin-4-yl]methyl-Caged Compounds as Ultrafast and Effective Long-Wavelength Phototriggers of 8-Bromo-Substituted Cyclic Nucleotides, Chem Bio Chem 4 434-442 (2003)) and is cleaved by visible light (U.S. patent application Ser. No. 11/402,715) (which are incorporated by reference herein with respect to such disclosures). Coumarin-based photolabile linkages are also available for linking to aldehydes and ketones (Lu et al., Bhc-diol as a photolabile protecting group for aldehydes and ketones, Org. Lett. 5 2119-2122 (2003)). Closely related analogues, such as BHQ, are also suitable (Fedoryak et al., Brominated hydroxyquinoline as a photolabile protecting group with sensitivity to multiphoton excitation, Org. Lett. 4 3419-3422 (2002)). Another suitable photocleavable group comprises the pHP group (Park and Givens, J. Am. Chem. Soc. 119:2453 (1997), Givens et al., New Phototriggers 9. p-Hydroxyphenacyl as a C-Terminal Photoremovable Protecting Group for Oligopeptides, J. Am. Chem. Soc. 122 2687-2697 (2000); Zhang et al., J. Am. Chem. Soc. 121 5625-5632, (1999); Conrad et al., J. Am. Chem. Soc. 122 9346-9347 (2000); Conrad et al., Org. Lett. 2 1545-1547 (2000)). A ketoprofen derived photolabile linkage is also suitable (Lukeman et al., Carbanion-Mediated Photocages: Rapid and Efficient Photorelease with Aqueous Compatibility, J. Am. Chem. Soc. 127 7698-7699 (2005)). The foregoing are incorporated by reference herein with respect to their disclosure of photocleavable groups.

In certain exemplary embodiments shown in the examples, the photocleavable group is di-methoxy nitro phenyl-ethyl or DMNPE.

As discussed above, a photocleavable group is one whose covalent attachment to a drug molecule is reversed (cleaved) by exposure to light of an appropriate wavelength. In one aspect, release of the drug molecule occurs when the conjugate is subjected to ultraviolet light. For example, photorelease of the drug molecule may occur at a wavelength ranging from about 200 to 380 nm (the exact wavelength or wavelength range will depend on the specific photocleavable group used, and could be, for example, 200, 210, 220, 230, 240, 250, 260, 270, 280, 290, 300, 310, 320, 330, 340, 350, 360, 370, or 380 or some range therebetween). In another aspect, release of the drug molecule occurs when the conjugate is subjected to visible light. For example, photorelease of the drug molecule may occur at a wavelength ranging from about 380 to 780 nm (the exact wavelength or wavelength range will depend on the specific photocleavable group used, and could be, for example, 380, 400, 450, 500, 550, 600, 650, 700, 750, or 780, or some range therebetween). In still another aspect, release of the drug molecule occurs when the conjugate is subjected to infrared light. For example, photorelease of the drug molecule may occur at a wavelength ranging from about 780 to 1200 nm (the exact wavelength or wavelength range will depend on the specific photocleavable group used, and could be for example, 780, 800, 850, 900, 950, 1000, 1050, 1100, 1150, or 1200, or some range therebetween). In general, longer wavelengths are preferred because they provide for greater tissue penetration and generally exhibit less toxicity. To avoid premature photorelease of the drug molecule, the depot may be shielded from background/ambient light using any suitable device, such as a patch, bandage, band, and the like.

In one aspect, the photocleavable group may be a diazo-azide. For example, the photocleavable functional group may be defined according to:

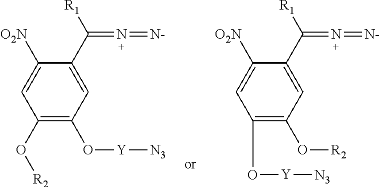

wherein R₁ is H or alkyl (preferably a C₁-C₆ alkyl); R₂ is H or alkyl (preferably a C₁-C₆ alkyl) and Y is a linker chain (preferably a linker chain comprising about 1 to 100 atoms). The linker may comprise C, N, O, S, and/or P atoms, and may comprise 5, 10, 15, 20, 25, 30, 35, 40, 45, 50, 55, 60, 65, 70, 75, 80, 85, 90, 95, or 100 atoms. Exemplary linkers include alkyl or polyether groups.

In another aspect, the photocleavable group may be a diazo-alkyne. For example, the photocleavable functional group may be defined according to:

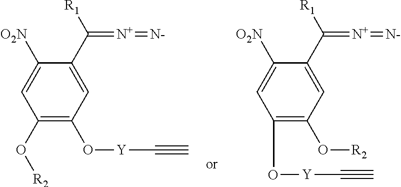

wherein R₁ is H or alkyl (preferably a C₁-C₆ alkyl); R₂ is H or alkyl (preferably a C₁-C₆ alkyl); and Y is a linker chain (preferably a linker chain comprising about 1 to 100 atoms). The linker may comprise C, N, O, S, and/or P atoms, and may comprise 5, 10, 15, 20, 25, 30, 35, 40, 45, 50, 55, 60, 65, 70, 75, 80, 85, 90, 95, or 100 atoms. Exemplary linkers include alkyl or polyether groups.

In one aspect, the photocleavable group used for crosslinking in this embodiment may be a bifunctional or multifunctional photocleavable group such that photolysis occurs at two or more places in the linker. In some aspects, the photocleavable group may be constructed as a dimer, trimer, or other -mer such that the “mer” units forming the photocleavable group are each photocleavable.

In one aspect, the photocleavable group may be a diazo-multimer. For example, the photocleavable functional group may be defined according to:

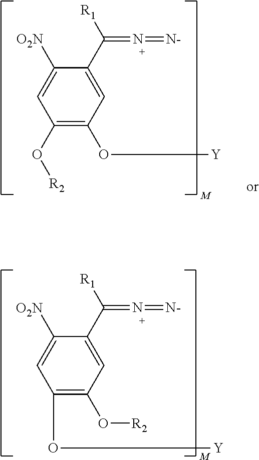

wherein R₁ is H or alkyl (preferably a C₁-C₆ alkyl); R₂ is H or alkyl (preferably a C₁-C₆ alkyl); and Y is a linker chain (preferably a linker chain comprising about 1 to 100 atoms); and M is an integer (preferably 2, 3, 4, or 5). The linker may comprise C, N, O, S, and/or P atoms, and may comprise 5, 10, 15, 20, 25, 30, 35, 40, 45, 50, 55, 60, 65, 70, 75, 80, 85, 90, 95, or 100 atoms. Exemplary linkers include alkyl or polyether groups.

In another aspect, the photocleavable group may be a carbonate-multimer. For example, the photocleavable functional group may be defined according to:

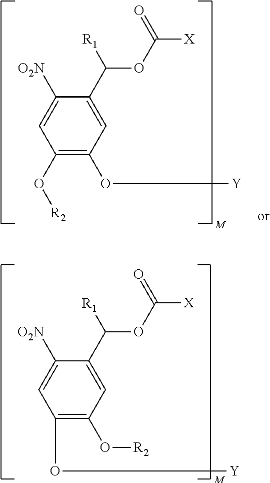

wherein R₁ is H or alkyl (preferably a C₁-C₆ alkyl); R₂ is H or alkyl (preferably a C₁-C₆ alkyl); X is a leaving group (such as N-hydroxyl succinimide); Y is a linker chain (preferably a linker chain comprising about 1 to 100 atoms); and M is an integer (preferably 2, 3, 4, or 5). The linker may comprise C, N, O, S, and/or P atoms, and may comprise 5, 10, 15, 20, 25, 30, 35, 40, 45, 50, 55, 60, 65, 70, 75, 80, 85, 90, 95, or 100 atoms. Exemplary linkers include alkyl or polyether groups.

Drug Molecules

The photocleavable drug conjugates of the present invention comprise one or more drug molecules. In general, the term “drug” as used herein refers to any substance that alters the physiology of a patient. The term “drug” may be used interchangeably herein or in the art with the terms “biologically active agent,” “therapeutic agent,” and “active pharmaceutical ingredient” or prodrug thereof as known in the art. Thus, the “drug” that is photoreleased from the conjugate may be a drug, drug precursor or modified drug that is not fully active or available until converted in vivo to its therapeutically active or available form.

The drug may include small molecule compounds, peptides, proteins, or any other medicament or medicine used in the treatment or prevention of a disease or condition. Representative non-limiting classes of drugs useful in the present invention include those falling into the following therapeutic categories: ACE-inhibitors; anti-anginal drugs; anti-arrhythmias; anti-asthmatics; anti-cholesterolemics; anti-convulsants; anti-depressants; anti-diarrhea preparations; anti-histamines; anti-hypertensive drugs; anti-infectives; anti-inflammatory agents; anti-lipid agents; anti-manics; anti-nauseants; anti-stroke agents; anti-thyroid preparations; anti-tumor drugs; anti-tussives; anti-uricemic drugs; anti-viral agents; acne drugs; alkaloids; amino acid preparations; anabolic drugs; analgesics; anesthetics; angiogenesis inhibitors; antacids; anti-arthritics; antibiotics; anticoagulants; antiemetics; antiobesity drugs; antiparasitics; antipsychotics; antipyretics; antispasmodics; antithrombotic drugs; anxiolytic agents; appetite stimulants; appetite suppressants; beta blocking agents; bronchodilators; cardiovascular agents; cerebral dilators; chelating agents; cholecystokinin antagonists; chemotherapeutic agents; cognition activators; contraceptives; coronary dilators; cough suppressants; decongestants; deodorants; dermatological agents; diabetes agents; diuretics; emollients; enzymes; erythropoietic drugs; expectorants; fertility agents; fungicides; gastrointestinal agents; growth regulators; hormone replacement agents; hyperglycemic agents; hypnotics; hypoglycemic agents; laxatives; migraine treatments; mineral supplements; mucolytics; narcotics; neuroleptics; neuromuscular drugs; NSAIDS; nutritional additives; peripheral vasodilators; polypeptides; prostaglandins; psychotropics; renin inhibitors; respiratory stimulants; steroids; stimulants; sympatholytics; thyroid preparations; tranquilizers; uterine relaxants; vaginal preparations; vasoconstrictors; vasodilators; vertigo agents; vitamins; and wound healing agents.

The drug molecules may be polymers with one or more functional groups suitable for linking to the photocleavable group (for example, drug molecules containing one or more amine, carboxyl, or thiol groups), such as therapeutic peptides. Alternatively, functional groups may be added to drug molecules to facilitate linkage of the drug molecule to the photocleavable group.

The preferred drugs molecules used in the present invention are those which are very potent such that they require relatively small amounts for the desired therapeutic effect but also need the blood levels to be carefully controlled. The preferred drugs are also those which benefit from good control of release.

In one aspect, the drug molecule is a therapeutic peptide or protein, such as those described in Bossard et al., U.S. Patent Application No. 2011/0166063 and Ekwuribe, U.S. Pat. No. 6,858,580, which are incorporated by reference herein with respect to such disclosures. Preferred therapeutic peptides and proteins are selected from the group consisting of insulin; glucagon; calcitonin; gastrin; parathyroid hormones; angiotensin; growth hormones; secretin; luteotropic hormones (prolactin); thyrotropic hormones; melanocyte-stimulating hormones; thyroid-stimulating hormones (thyrotropin); luteinizing-hormone-stimulating hormones; vasopressin; oxytocin; protirelin; peptide hormones such as corticotropin; growth-hormone-stimulating factor (somatostatin); G-CSG, erythropoietin; EGF; physiologically active proteins, such as interferons and interleukins; superoxide dismutase and derivatives thereof; enzymes such as urokinases and lysozymes; and analogues or derivatives thereof. In another aspect, the therapeutic peptide or protein is selected from the group consisting of human growth hormone, bovine growth hormone, growth hormone-releasing hormone, an interferon, interleukin-1, interleukin-II, insulin, calcitonin, erythropoietin, atrial natriuretic factor, an antigen, a monoclonal antibody, somatostatin, adrenocorticotropin, gonadotropin releasing hormone, oxytocin, vasopressin, analogues, or derivatives thereof.

In another aspect, the drug molecule is an anti-diabetic agent already in the clinical practice or in the pipeline of development. The anti-diabetic drug molecules are broadly categorized herein as insulin/insulin analogs and non-insulin anti-diabetic drugs. The non-insulin anti-diabetic drugs may include, but not limited to, insulin sensitizers, such as biguanides (e.g., metformin, buformin, phenformin, and the like), thiazolidinedione (TZDs; e.g., pioglitazone, rivoglitazone, rosiglitazone, troglitazone, and the like), and dual peroxisome proliferator-activated receptor agonists (e.g., aleglitazar, muraglitazar, tesaglitazar, and the like). The non-insulin anti-diabetic drugs may also include, but not limited to, secretagogues, such as sulfonylureas (e.g., carbutamide, chlorpropamide, gliclazide, tolbutamide, tolazamide, glipizide, glibenclamide, gliquidone, glyclopyramide, glimepiride, and the like), meglitinides (e.g., nateglinide, repaglinide, mitiglinide, and the like), GLP-1 analogs (e.g., exenatide, liraglutide, albiglutide, taspoglutide, and the like), and dipeptidyl peptidase 4 inhibitors (e.g., alogliptin, linagliptin, saxagliptin, sitagliptin, vildagliptin, and the like). Further, the non-insulin anti-diabetic drugs may include, but not limited to, alpha-glucosidase inhibitors (e.g., acarbose, miglitol, voglibose, and the like), amylin analog (e.g., pramlintide and the like), SGLT2 inhibitors (e.g., dapagliflozin, remogliflozin, sergliflozin, and the like), benfluorex, and tolrestat.

One preferred drug molecule is insulin. As used herein, the term insulin embraces analogues or derivatives thereof. Exemplary insulin compounds are described in Foger et al., U.S. Published Patent No. 2011/0144010, which is incorporated by reference with respect to such disclosures. In another aspect, the drug is insulin (or an analog or derivative thereof) in its hexameric form, typically in the presence of zinc. Another preferred drug molecule is glucagon.

In an exemplary aspect, the carboxyl functionalities found on insulin are able to form a photolabile bond with a photocleavable group having a DMNPE group. Upon photolysis, the carboxyl functionality is released from the DMNPE, generating native insulin. It will be appreciated that amine or other functional groups on insulin can be used to form a photolabile bond with the photocleavable group.

It will be appreciated that while the moiety may comprise some small molecule drugs (or prodrugs) having reactive functional groups, the invention is particularly well suited for crosslinking of peptides, proteins, nucleic acids, and other macromolecules. Peptides having about 10 to 500 amino acid residues (e.g., about 10, 20, 30, 40, 50, 60, 70, 80, 90, 100, 150, 200, 250, 300, 350, 400, 450, or 500 residues or some range therebetween) are most preferred.

Typically, the ratio of drug to the solubility modulating protein is about 95:5, 90:10, 80:20, 70:30, 60:40, 50:50, 40:60, 30:70, 20:80, 10:90, or 5:95 (wt:wt) or some range therebetween.

Combination or Multi-Drug Delivery

The compositions and depots of the present invention comprise one or more photocleavable drug conjugates. The photocleavable drug conjugate used in the composition may be comprised of different moiety types, different photocleavable group types, different drug molecule types, or a combination thereof. Thus, the compositions and depots of the present invention are well adapted to the administration of multiple drugs types.

In one aspect, the depot comprises a first photocleavable drug conjugate comprising a first moiety linked to a first photocleavable group which is in turn linked to a first drug molecule. The depot may also comprise a second photocleavable drug conjugate comprising a second moiety linked to a second photocleavable group which is in turn linked to a second drug molecule. The first and second moieties may be of the same or different type. The first and second photocleavable groups are of a different type, and the first and second drug molecules are of a different type. The excitation wavelength may be chosen so as to selectively excite and cleave the particular photocleavable groups. As a result, independent control of the release of the first drug and the second drug from the depot may be achieved.

As an example of the foregoing, the depot may comprise a first photocleavable drug conjugate comprising a non-polar or charged moiety linked to insulin via a NDBF group. The depot may also comprise a second photocleavable drug conjugate comprising a non-polar or charged moiety linked to glucagon via a NPE group. The depot may be irradiated with two different wavelengths (e.g., one that cleaves NDBF and another wavelength that cleaves NPE) either simultaneously or sequentially in order to control the release of the two drug molecules.

An exemplary situation in which the release of two different drugs with two different wavelengths would be useful is with insulin and glucagon. Insulin is the natural signal that stimulates cells to absorb glucose from the bloodstream, whereas glucagon is a signal that stimulates cells to release glucose into the bloodstream. As such, they form a pair that finely regulates blood sugar. In a diabetic patient both of these signals could be released from a photoactivated depot using different light wavelengths for each, allowing native-like control of blood sugar. The link of the photocleavable group to glucagon and insulin has additional advantages beyond the ability to control its release.

From the foregoing, it is contemplated that it is possible to photoreleasably attach multiple different drug molecule types and/or different photocleavable group types to the moieties, and then independently control the photorelease the drugs by selecting the excitation wavelength to match the corresponding photocleavable groups.

Depot Implantation

The photocleavable drug conjugate is generally designed to function as a drug depot. The photocleavable drug conjugate is preferably formulated in the composition of the invention that is suitable for implantation to form a depot beneath the skin of the patient, typically via cutaneous, subcutaneous, or intramuscular implantation. A drug depot comprising the drug conjugate may be implanted in a manner similar to currently used with native insulin.

There are a number of common locations within a patient that may be sites at which the drug depot may be implanted. For example, administration may be required in a patient's arms, shoulders, knees, hips, fingers, thumbs, neck, legs, abdomen, head, buttocks, feet, back, and/or spine.

In one aspect, the depot is located in the cutaneous region of the skin, for example, in the stratum germinativum and/or stratum spinosum of the epidermis. In another aspect, the depot is located in the dermis, for example in the papillary layer and/or the reticular layer. The patient may be implanted with a single depot or with an array of depots, e.g., such that smaller depots comprising the conjugate are implanted in a localized region. The location is preferably such that the tissue is sufficiently vascularized to permit distribution of the drug through the body. The location is also preferably such that the light is able to penetrate through the tissue to photorelease the drug from the conjugate.

The depot comprising the photocleavable drug conjugate of the present invention is generally implanted into the patient in need of delivery of the drug. The term “implantable” as utilized herein includes implantable through surgery, injection, or other suitable means. Typically, implantation is made cutaneously, subcutaneously, or intramuscularly using techniques generally known to those skilled in the art. In certain embodiments, the patient may implant the depot by methods similar to those used by patients to self-administer insulin.

The depot comprising the photocleavable drug conjugate is typically administered to the target site of the patient using a “cannula” or “needle” that can be a part of a drug delivery device, e.g., a syringe, a gun drug delivery device, or any medical device suitable for the application of a drug to a targeted organ or anatomic region. The cannula or needle of the drug depot device is designed to cause minimal physical and psychological trauma to the patient.

Cannulas or needles include tubes that may be made from materials, such as for example, polyurethane, polyurea, polyether(amide), PEBA, thermoplastic elastomeric olefin, copolyester, and styrenic thermoplastic elastomer, steel, aluminum, stainless steel, titanium, metal alloys with high non-ferrous metal content and a low relative proportion of iron, carbon fiber, glass fiber, plastics, ceramics or combinations thereof. The cannula or needle may optionally include one or more tapered regions. The cannula or needle may be beveled. The cannula or needle may also have a tip style vital for accurate treatment of the patient depending on the site for implantation. Examples of tip styles include, for example, Trephine, Cournand, Veress, Huber, Seldinger, Chiba, Francine, Bias, Crawford, deflected tips, Hustead, Lancet, or Tuohey. The cannula or needle may also be non-coring and have a sheath covering it to avoid unwanted needle sticks. The dimensions of the hollow cannula or needle, among other things, will depend on the site for implantation.

The patient of the present invention is preferably an animal (for example, warm-blooded mammal) and may be either a human or a non-human animal. Exemplary non-human animals include but are not limited to non-human primates, rodents, farm animals (for example, cattle, horses, pigs, goats, and sheep) and pets (for example, dogs, cats, ferrets, and rodents). The patient is typically a mammal. The term “mammal” refers to organisms from the taxonomy class “mammalian,” including but not limited to humans, chimpanzees, apes, orangutans, monkeys, rats, mice, cats, dogs, cows, horses, etc.

Certain embodiments, the depot is an insoluble and solid or semi-solid (gel) use for delivery of drug to the body of a patient. The depot generally forms a mass to facilitate implantation and retention in a desired site of the patient. The depot can also be a liquid at room temperature that turns into a gel at body temperature, i.e., a thermosensitive gel.

The depot may have different sizes, shapes, and configurations. There are several factors that may be taken into consideration in determining the size, shape, and configuration of the depot. For example, both the size and shape may allow for ease in positioning the drug depot at the target tissue site that is selected as the implantation or injection site. In addition, the shape and size of the depot should be selected so as to minimize or prevent the drug depot from moving after implantation or injection. In various aspects, the drug depot may be shaped like a sphere, a cylinder such as a rod or fiber, a pellet, a flat surface such as a disc, film or sheet (e.g., ribbon-like) or the like. The drug depot may also have an amorphous or undefined shape. Flexibility may be a consideration so as to facilitate placement of the drug depot. The overall design of a suitable drug depot is well known to those skilled in the art. Exemplary sizes of the depot can be as very small, for example low μm size (such as 1l m or 0.001 mm), small (0.001 mm to 1 mm), intermediate (1 mm to 5 mm) or larger (5 mm to 10 mm), and can be any value or range therebetween. In certain embodiments, the depot can have a volume up 100 μl, although other volumes are contemplated. The nonpolar tag materials end up as suspensions of particles, and the charge tag materials are totally homogenous until injected. The charge tag materials may also be a suspension of particles if the concentration is above the solubility limit. This enables the formation of small depots that minimize pain to the patient.

The photocleavable drug conjugate of the present invention is formulated into a depot. It will be appreciated to those skilled in the art that the depot may optionally contain inactive materials such as saline, buffering agents and pH adjusting agents such as potassium bicarbonate, potassium carbonate, potassium hydroxide, sodium acetate, sodium borate, sodium bicarbonate, sodium carbonate, sodium hydroxide or sodium phosphate; degradation/release modifiers; drug release adjusting agents; emulsifiers; preservatives such as benzalkonium chloride, chlorobutanol, phenylmercuric acetate and phenylmercuric nitrate, sodium bisulfate, sodium bisulfite, sodium thiosulfate, thimerosal, methylparaben, polyvinyl alcohol and phenylethyl alcohol; solubility adjusting agents; stabilizers; and/or cohesion modifiers. If the depot is to be placed in the spinal area, the depot may comprise sterile preservative free material.

In one aspect, the drug depot includes one or more viscosity enhancing agents, such as, for example, hydroxypropyl cellulose, hydroxypropyl methyl cellulose, hydroxyethyl methylcellulose, carboxymethylcellulose and salts thereof, Carbopol, poly-(hydroxyethylmethacrylate), poly-(methoxyethylmethacrylate), poly(methoxyethoxyethyl methacrylate), polymethylmethacrylate (“PMMA”), methylmethacrylate (“MMA”), gelatin, polyvinyl alcohols, propylene glycol; PEG 200, PEG 300, PEG 400, PEG 500, PEG 600, PEG 700, PEG 800, PEG 900, PEG 1000, PEG 1450, PEG 3350, PEG 4500, PEG 8000, or combinations thereof.

Drug Cleavage

Once implanted into the patient, the depot comprising the photocleavable drug conjugate provides for immediate and/or controlled release of the drug using light activation to cleave the drug molecule from the solubility modulating portion. Because the cleavage can occur at the linkage between the drug molecule and the photocleavable group, the drug molecule can be released in its native form without addition of other groups or other modifications.

Upon exposure to light of the appropriate wavelength, the drug molecule is cleaved from the drug conjugate via photolysis, thereby releasing the drug from the conjugate. The desired drug release from the conjugate may also be modulated by controlling the intensity of the light exposure, duration of the light exposure, and the location of implantation.

In one aspect, irradiation is accomplished by a light source located external to the patient. The external light source may be possibly worn like a band, patch, or bandage over the depot site. In such embodiment, the external light source may also serve as a shield from ambient light. The irradiation to promote photorelease of the drug can be provided by a variety of sources including, but not limited to light emitting diodes (LEDs), lasers, pens, and even incandescent, fluorescent, or ultraviolet bulbs. Various phototherapy devices are known in the art and could be readily adapted for use in the present invention. For example, there are many commercially phototherapy devices uses for the treatment of psoriasis, wound repair, and other skin diseases (such as those manufactured by TheraLight, Inc.) which could be modified for use in the present invention. Other exemplary phototherapy devices include, but are not limited to those described in Passy et al., U.S. Pat. No. 7,513,906; Parker et al., U.S. Pat. No. 7,686,839; Hubert et al., U.S. Pat. No. 7,878,203; Gertner et al. U.S. Published Application No. 2006/0206173; Lewis, U.S. Published Application No. 2008/0269849; Holloway et al. U.S. Published Application No. 2004/0166146; all of which are incorporated by reference herein with respect to such disclosures.

The light-emitting device provides irradiation to the skin surface of the patient in the area overlying the depot sufficient penetrate the tissue overlying the conjugate. The light results in the photorelease of the desired amount of drug molecules from the conjugate. Broadly speaking, the light-emitting device thus provides for “transdermal” irradiation of the depot although the depot may be located cutaneously, subcutaneously, or intramuscularly, as generally described herein. In certain embodiments, the light source provides light of the same wavelength as ambient light, but a higher intensity.

The drug in the depot may be released by transdermal irradiation in response to a physiological signal. For example, when the drug is insulin, blood sugar information provided by the patient through traditional finger sticks or by one of the non-invasive monitoring methods being developed in the field can be used.

The light-emitting device may include a controller or computer programmed to irradiate the skin of the patient in a number of different ways. The irradiation may be provided at fixed or variable intervals. For example, for drugs requiring conventional twice per day (“BID”) or three times per day (“TID”) dosing, the light emitting device may be programmed to provide irradiation two or three times per day, respectively. Alternatively, the light emitting device may be coupled to a sensor which measures a variable dependent upon the drug concentration in the body and then provides feedback to the light emitting device to control the light irradiation. For example, in the case of insulin, the light emitting device may be coupled to a sensor which measures the amount of insulin in the blood stream or other parameter (most likely the blood glucose concentration). The light emitting device may be programmed to irradiate the skin of the patient in accordance with that feedback loop. In short, the amount of light generated from the light emitting device can be periodically or continually modulated depending on the desired outcome. Sensors and other devices for measuring the dependent variable of interest (such as blood glucose) are generally described in Jennewine, U.S. Published Application No. 2009/0054750; Hayter et al., U.S. Published Application No. 2009/0164239; Blomquist, U.S. Published Application No. 2008/0172031; Talbot et al., U.S. Published Application No. 2005/0065464; all of which are incorporated by reference herein with respect to such disclosures.

The photocleavable drug conjugate of the present invention may provide immediate release of the drug, sustained release of the drug, or a combination thereof. For example, in general, immediate release of the drug may occur by irradiation of the photocleavable drug conjugate with appropriate light such that the drug is released from the photocleavable drug conjugate. This generally results into the introduction of the active drug into the body and that such that the drug is allowed to dissolve in or become absorbed at the location to which it is administered, with little or no delaying or prolonging of the dissolution or absorption of the drug.

As another example, once cleaved from the photocleavable drug conjugate, the drug may also undergo sustained release. In general, sustained release (also referred to as extended release or controlled release) encompasses ability of the photocleavable drug conjugate to continuously or continually release of the drug over a predetermined time period as a result of controlled irradiation with light. That is, the depot comprising photocleavable drug conjugate comprises a reservoir of drug molecules in which the release of the drug molecules from the conjugate may be photocontrolled over an extended period of time (e.g., days, weeks, or months).

In one aspect, the present invention overcomes the problem associated with conventional drug delivery whereby frequent injections of the drug, such as insulin, are needed. For example, a patient may require a total daily dose of insulin of about 1 to 100 IU per day (e.g., about 1, 2, 3, 4, 5, 6, 7, 8, 9, 10, 20, 30, 40, 50, 60, 70, 80, 90, 100 IU per day), and typically about 0.1 to 2 IU/kg/day (e.g., about 0.1, 0.2, 0.3, 0.4, 0.5, 0.6, 0.7, 0.8, 0.9, 1, 1.0, 1.1, 1.2, 1.3, 1.4, 1.5, 1.6, 1.7, 1.8, 1.9, 2 IU/kg/day). This may be a dose of about 1 to 4 mg of insulin per day. In the present invention, the depot may contain a supply of insulin that lasts for several days, weeks, or even months, including a supply for 2, 3, 4, 5, 6, 7, 8, 9, 10, 11, 12, 13, 14, 15, 20, 25, 30, 35, 40, 45, 50, 55 or 60 days. It is contemplated that in one aspect, an entire one-month, or even two-month, supply or more of insulin could be deposited in the drug depot in a single injection in a volume equivalent to a single dose of traditional insulin. This dramatically reduces the number of injections needed to control a patient's disease, that is, there may be as much as a 50-, 100-, or even 200-fold reduction in the injection number. In another aspect, the present invention overcomes the problem associated with conventional insulin use whereby there is significant variability of blood sugar levels. In the present invention, there is a potential for rapid (e.g., real time, minute-by-minute, or hour-by-hour) correction of blood sugar levels through the non-invasive and continuously variable release of insulin with light. In one aspect, native like, rock-level blood sugar levels of a non-diabetic could potentially be obtained.

Further, when the drug molecule is insulin, there is a potential for rapid (e.g., real time or even minute by minute) correction of blood sugar levels through the non-invasive and continuously variable release of insulin with light. In one aspect, native like, rock-level blood sugar levels of a non-diabetic could potentially be obtained.

Drug Conjugate Synthesis

In certain aspects of the invention, the overall synthetic scheme for the drug conjugates having a peptide moiety (non-polar or charged) comprises the following steps:

a) Solid phase synthesis of hydrophobic peptide

b) Reaction of peptide with DMNPE derivative containing ketone group

c) Conversion of ketone to hydrazone

d) Cleavage of hydrazone from resin

e) Conversion of hydrazone to diazo group

f) Reaction of diazo group with insulin to make final product.

It was surprisingly found that the conversion of the hydrazone to a diazo group was a very difficult step in the process. It was discovered that a side reaction was forming an unproductive azine product (a dimer-like molecule of the hydrazone). It is believed the azine was being formed due to self-association of the hydrazone, driving the azine formation. It is further believed this problem occurs with peptide moieties because the peptides can form associations such as beta sheets. By using a much lower concentration of hydrazone, the reaction effectively made the diazo, and allowed it to react with a carboxylic acid. It was further determined that reacting the hydrazone immediately after synthesis helped improve the yield of the diazo. Preferably the hydrazone is reacted within 24 hours after synthesis.

Generally hydrazone concentrations above 100 mM can be used in the conversion of a hydrazone to a diazo group. However, when the moiety attached to the photocleavable group comprised a peptide, it was surprisingly found that use of a much smaller concentration of hydrazone produced better yields, such as 8.28 mM, 11.04 mM, 12.2 mM, and 16.56 mM, which are 10 to 20 times less than standard concentrations. Thus, it is expected that hydrazone concentrations of 50 mM or less, or 25 mM, 20 mM, 15 mM, or 10 mM or less, or concentrations that are 50, 25, 20, 15, or 10 times less than would be standard, would be suitable for reactions using moieties with peptide or amino acid groups.

Similar processes can be used to form drug conjugates with moieties that do not comprise a peptide or amino acid.

In some aspects, the conjugates of the present invention may be synthesized using bioorthogonal coupling reactions, which may include, but are not limited to the chemistry found in Native Chemical Ligation (“NCL”) and Expressed Protein Ligation (“EPL”), carbonyl ligations, Diels-Alder reactions, Pd- and Rh-catalyzed ligations, decarboxylative condensations, thioacid/azide ligations, maleimide/thiol pairs, aziridine ligations, the Staudinger ligation, and the Sharpless-Huisgen cycloaddition. These reactions are often cited as examples of “click chemistry,” a term used in the art to refer to chemical reactions that are specific, high yielding, and tolerant of functional groups.

Additional Considerations

The photocleavable drug conjugate of the present invention may be modified in various ways. For example, one or more linkers may be used to vary the distance between moiety and photocleavable group. Likewise, one or more linkers may be used to vary the distance between photocleavable group and the drug molecule. The linker length may be 1, 2, 3, 4, 5, 6, 7, 8, 9, 10, 11, 12, 13, 14, 15, 16, 17, 18, 19, 20, 22, 24, 26, 28, 30, 32, 34, 36, 38, 40, 42, 44, 46, 48, or 50 atoms (e.g., carbons) long. The linker may be comprised of carbon, nitrogen, oxygen, sulfur, and phosphorous atoms. For example, the linker may be an alkyl or contain ether, ester, and/or amines groups.

Other Crosslinking Groups

In one aspect, the photocleavable drug conjugate comprises photocleavable groups which may also be cleaved using other mechanisms. Preferably, the linker is cleaved under mild conditions, i.e., conditions within a cell under which the activity of the drug is not affected. Examples of suitable cleavable linkers include disulfide linkers, acid labile linkers, peptidase labile linkers, and esterase labile linkers. Disulfide containing linkers are linkers cleavable through disulfide exchange, which can occur under physiological conditions. Acid labile linkers are linkers cleavable at acid pH. For example, certain intracellular compartments, such as endosomes and lysosomes, have an acidic pH (pH 4-5), and provide conditions suitable to cleave acid labile linkers. Peptidase labile linkers can be used to cleave certain peptides inside or outside cells. Photolysis may result the release of a smaller aggregate of the crosslinked drug conjugate. In turn, these smaller aggregates may form even smaller aggregates or individual drug molecules as generally described herein.

For example, the photocleavable group may comprise a carbamate linkage to the drug molecule. The carbamate can be both photolyzed to release the drug and can also be cleaved by esterases to produce native insulin. If an aggregate of crosslinked drug molecules is photocleaved and released from the main portion of the drug depot, the drug molecules in this smaller aggregate may still be released by esterases within the body as the aggregate is absorbed by and/or distributed therein. However, in general, this esterase action will be limited when the carbamate link resides within the main depot since there will be limited access to esterases at the depot site.

The present invention provides new drug conjugate molecules and methods for using the molecules. The molecules comprise solubility modulating portions that modify drug molecules into insoluble materials that can be released from insoluble depots upon exposure to light, through the cleavage of the hydrophobic tag. The drug conjugates can be formulated into injectable particles that form photoactivated depots of drug molecules such as insulin. The drug conjugates of the present invention address several of the limitations of previous light activated depot materials, namely density and the need for polymers. This increases the utility of the drug conjugates by increasing the potential duration of action, decreasing the amount of light needed to release, and by allowing byproducts of photolysis, small tags, to be much more efficiently cleared from the system.

The present invention will now be described with reference to the following examples. It should be appreciated that these examples are for the purposes of illustrating aspects of the present invention, and do not limit the scope of the invention as defined by the claims.

### Example 1. Synthesis of Drug Conjugate Comprising Insulin and Cyclododecyl Amine

An exemplary synthetic scheme for a drug conjugate of the present invention comprising insulin as the drug molecule and cyclododecyl amine as the non-polar moiety is depicted in FIG. 4. An exemplary synthesis was carried out as follows:

Synthesis of Keto-Ester

63.2 mmol (10.5 g) of acetovanillone, 69.2 mmol (13.5 g) tert-butylbromoacetate and 104 mmol (14.4 g) of potassium carbonate were added to 75 ml of dimethylformamide and stirred for 48 hours at room temperature. After 48 hours, water was added to the mixture until all salts were dissolved. This mixture was then partitioned between ethyl acetate and water. Pooled ethyl acetate fractions were washed with saturated NaCl, dried by addition of anhydrous magnesium sulfate. Ethyl acetate fraction was collected by filtration (to remove magnesium sulfate) and dried on a rotovap. This yields keto-ester as a white solid compound. Material was confirmed by MS and NMR.

Synthesis of Nitro-Keto-Acid (NKA)

3 ml of 70% nitric acid and 2 ml of acetic anhydride were cooled below 0 degrees C. Nitration mixture was first prepared by addition of 2 ml of acetic anhydride to 3 ml of 70% nitric acid on an ice bath to maintain the temperature below 0 degrees C.

3.6 mmol (1 g) of ketoester was dissolved in 3 ml (or slightly excess) of acetic anhydride. This solution was slowly added (dropwise) to the nitration mixture on ice bath. Care should be taken that the temperature should not rise. Reaction mixture was stirred for 2 hours on ice bath, and for another 4 hours at room temperature. A light-yellow precipitate may or may not be seen in the reaction mixture.

Reaction mixture was poured on to ice in a beaker and allowed to stand at 4 degrees overnight. Product can then be obtained by filtering the mixture and washing it extensively with cold water. Material may be recrystallized in MeOH/water mixture. Compound was characterized by MS and NMR.

Synthesis of CD-NKA

744 μmol (200 mg) NKA, 1.5 mmol (272.4 mg) cyclododecylamine (CD) and 1.5 mmol (227.6 mg) 1-hydroxybenzotriazole hydrate were dissolved in 4.5 ml DMF. 1.2 mmol (238.4 mg) 1-ethyl-3-(3-dimethylaminopropyl)-carbodiimide hydrochloride was added to this mixture and reaction was continued for 15 hours. Product was obtained by partitioning the mixture between ethyl acetate and acidified water (1 N HCl) fractions. Ethyl acetate fractions were pooled, washed with saturated sodium chloride and dried by addition of anhydrous magnesium sulfate. Magnesium sulfate was removed by filtration, ethyl acetate fraction was evaporated to yield the solid product. Compound was characterized by MS and NMR.

Synthesis of CD-Hydrazone

This is a sealed tube reaction. 295 μmol (128 mg) of hydrazone was dissolved in 8 ml of Acetonitrile:Ethanol (1:1) mixture. 441.6 μmol (26.4 μl) glacial acetic acid was added and mixed with the solution. 5.88 mmol (284.8 μl) hydrazine monohydrate was added to this mixture, reaction vial was sealed and the reaction was carried out at 90 degrees C. for 4 hours. Resulting solution was dissolved in dichloromethane (as little as possible) and run on a silica column to purify the hydrazone. Mobile phase is a mixture DCM:Methanol (95:5). Fractions were collected, dried and analysed by MS and NMR. HPLC revealed purity of sample.

Synthesis of Diazo

Purified, dried CD-hydrazone was dissolved in least amount of anhydrous DMSO. The concentrated solution was quantitated using UV-spectroscopy at 345 nm using an extinction coefficient of 4470 M⁻¹ cm⁻¹. Final concentration was adjusted to 0.101 M CD-hydrazone by diluting the original solution with anhydrous DMSO.

5.37 μmol (53.7 μl) was transferred into a 1.5 ml Eppendorf tube and 149.1 mol (13 mg) manganese dioxide was added and shaken vigorously for 45 minutes. Reaction mixture was immediately centrifuged at 12000 RPM for 5 min to remove manganese dioxide. Formation of diazo can be identified by a characteristic red color. 50 μl supernatant was collected into another Eppendorf tube. Manganese dioxide is washed with additional 750 μl of DMSO to recollect any trapped diazo. Solutions are pooled to make 800 μl diazo solution. Note that this diazo should be immediately reacted with Insulin as it is unstable. Compound characterized only by UV-Visible absorbance. Diazo will have a characteristic absorbance at 450 nm, unlike ketone and hydrazone that have absorbance only at 345 nm.

Synthesis of CD-Insulin

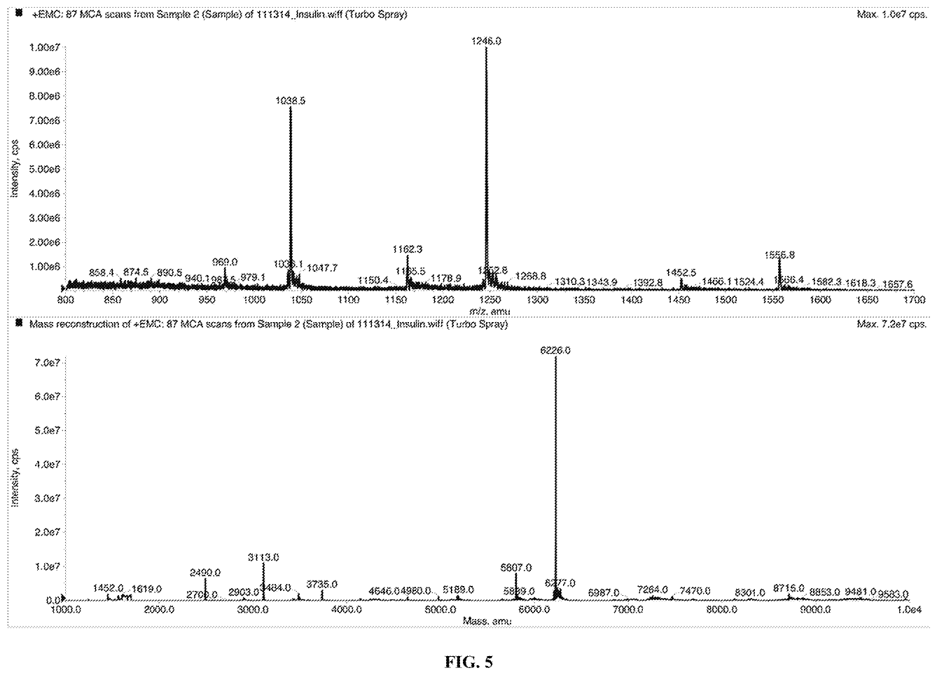

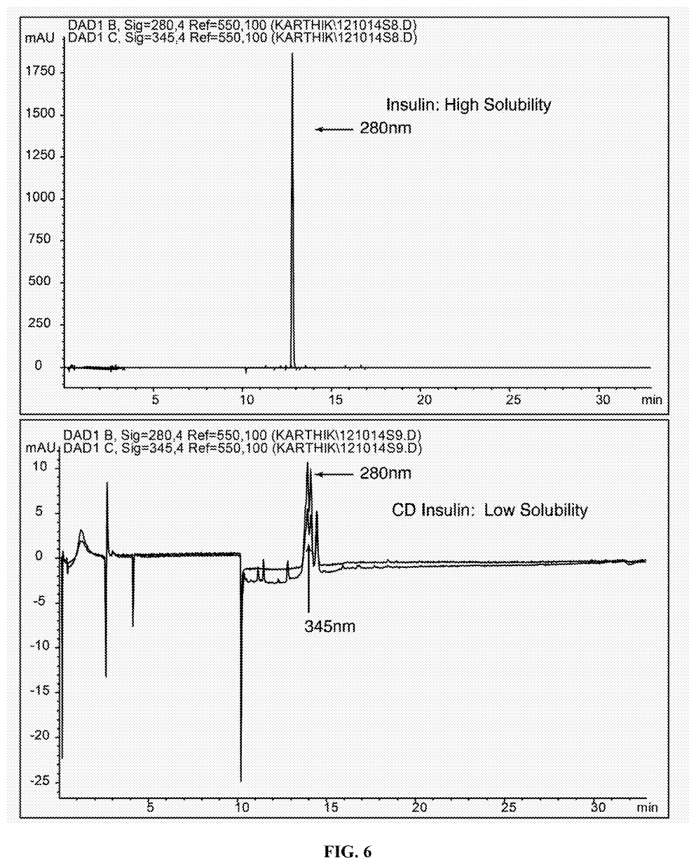

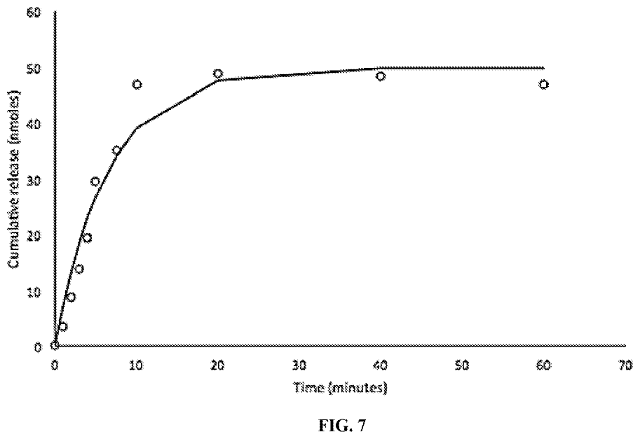

4.37 μmol (25 mg) of Insulin was weighed and dissolved in 800 μl anhydrous DMSO. 800 μl diazo solution (from previous reaction) was immediately added to the Insulin solution. Diazo was allowed to react for 24 hours. Reaction progress may be monitored on HPLC with a C18 column. CD-Insulin resolves on C18 column with its distinct retention time and may be purified as it elutes out of the column. Material can be identified with UV-spectroscopy and MS, as shown in FIG. 5. Solubility studies confirmed a significant drop (75 fold) in solubility of CD-insulin vs. unmodified insulin, as shown if FIG. 6. Solubility of insulin is 4.11 mg/ml (0.708 mmoles/lit). Solubility of CD-insulin is 0.56 mg/ml (0.00898 mmoles/lit). Release of native, unmodified insulin after photolysis is demonstrated in FIG. 7 (graph of insulin released into solution v. amount of irradiation time; k=0.154 min-).

### Example 2. Synthesis of Drug Conjugate Comprising Insulin and Val-Pro-Ile

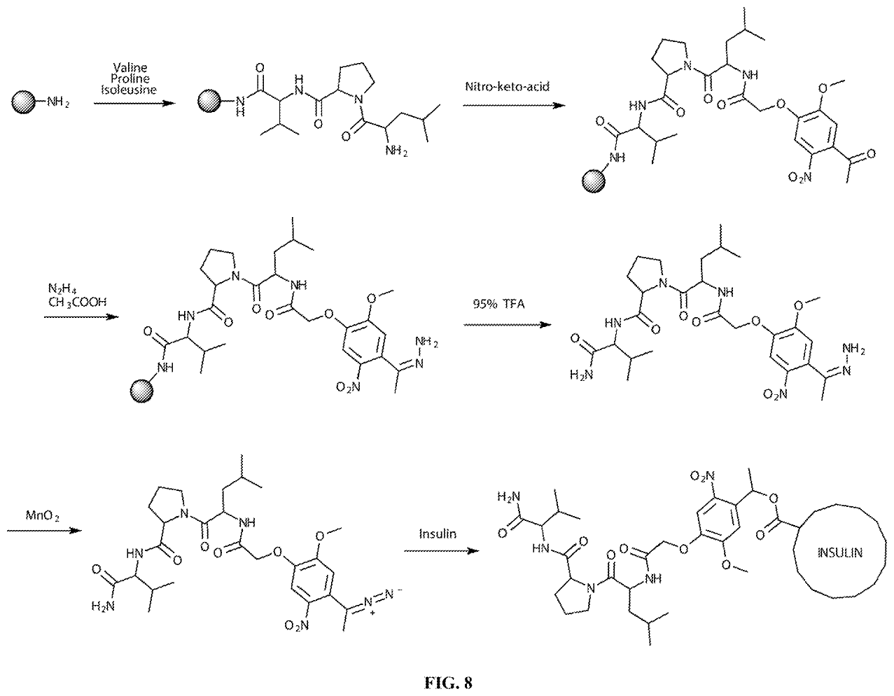

An exemplary synthetic scheme for a drug conjugate of the present invention comprising insulin as the drug molecule and a valine-proline-isoleucine as the non-polar moiety is depicted in FIG. 8. An exemplary synthesis was carried out as follows:

Synthesis of Keto-Ester

As described above in Example 1.

Synthesis of NKA

As described above in Example 1.

Synthesis of Val-Pro-Ile-NKA

Val-Pro-Ile-NKA was made by solid phase synthesis.

CHEMMATRIX® Rink amide resin was used for this purpose. Dry resin (quantitated) was weighed and suspended in DCM, in a peptide synthesizer. DCM was removed by vacuum, and washed with excess of NMP five times.

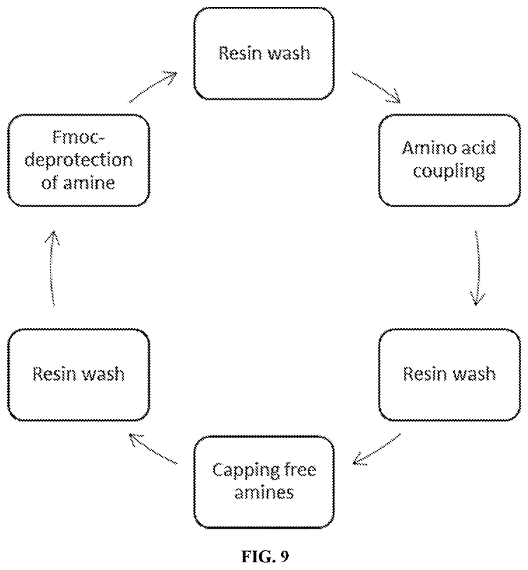

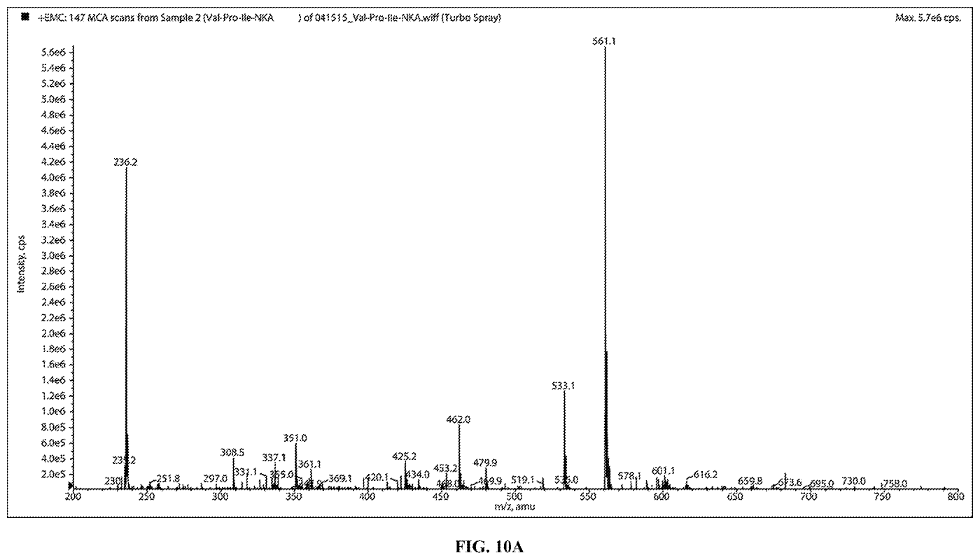

Synthesis is carried out as shown in FIG. 9. Each wash step is performed by washing with NMP. Wash step is repeated 5 times to ensure that the resin is free from any reagents used in previous steps. Resin is finally suspended in a volume such that the total amines (on resin) in suspension is about 60 mM.

Amount of Fmoc-amino acid taken is 5 times excess of the amines present on resin, i.e. will have a concentration of 300 mM in solution. Fmoc-Amino acid is first activated before coupling it on to the resin. Activation is carried out by dissolving amino acid in NMP and adding a 1:1 molar equivalents of HATU and 1:2 molar equivalents of DIEA. This mixture is allowed to stand for about 10-15 minutes and immediately added to the Rink amide resin. Final conditions of the coupling is shown Table 1 below. Coupling is carried out for 3 hours.

TABLE 1

| Reagent | Concentration |
|---|---|
| Amines (from Resin) | 60 mM |
| Fmoc-Amino acid | 300 mM |
| HATU | 300 mM |
| DIEA | 600 mM |

Resin is washed before capping free amines. Capping is performed using a solution of 10% acetic anhydride and 5% DIEA in NMP. This is performed for five minutes and the mixture is removed from resin by vacuum.

Resin is washed thoroughly (˜5 times) with NMP before removing the Fmoc group. Amines are deprotected by suspending the resin in NMP with 20% piperidine solution. This solution was removed after 5 minutes, and analysed on UV spec for characteristic fluorenyl group absorbance at 301 nm. Fresh 20% piperidine solution is again added to the resin and this process is repeated until 301 nm absorbance of the solution goes to 0 (or minimum), which is an indication that all the Fmoc groups have been removed in previous step.

Fmoc deprotection yields free amines and the next amino acid or carboxylic acid can be coupled after activation. This process is repeated with respective amino acid at each cycle until the desired peptide is obtained.

Product may be removed from a small amount of resin by treating it with 95% TFA, 5% water. TFA solution containing desired peptide is collected and washed with cold ether. Peptide is completely dried on rotovap and analysed by LCMS to confirm its mass.

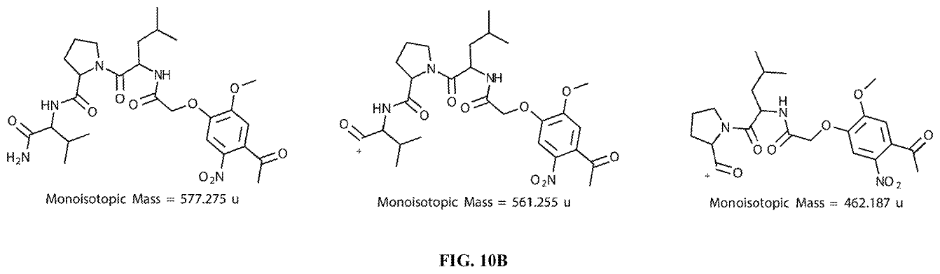

Val-Pro-Ile-NKA is synthesized per the procedure mentioned above. Coupling cycles Material is stored on resin until used for next reaction. FIG. 10 shows the MS data demonstrating the synthesis of the ketone intermediate.

Val-Pro-Ile-Hydrazone Synthesis

Val-Pro-Ile-NKA (ketone) was converted to the hydrazone on resin. Resin was initially washed with a solvent mixture of NMP:ethanol (1:1).

Reaction was carried out in a sealed siliconized glass reaction vessel. 250 mol of ketone (assuming 100% coupling on resin, 250 μmol of resin) was suspended in 9 ml of 1:1 NMP:ethanol solvent mixture. 1250 μmol (74 μl) glacial acetic acid was added and mixed gently and thoroughly. 40 times excess hydrazine monohydrate (10 mmol, 484.3 μl) was added to this mixture and reaction vessel was sealed tightly. This was shaken on an Eppendorf Thermomixer at 60 degrees C. Reaction was continued overnight. Resin was then thoroughly washed with NMP and DCM. Resin was allowed to dry completely.

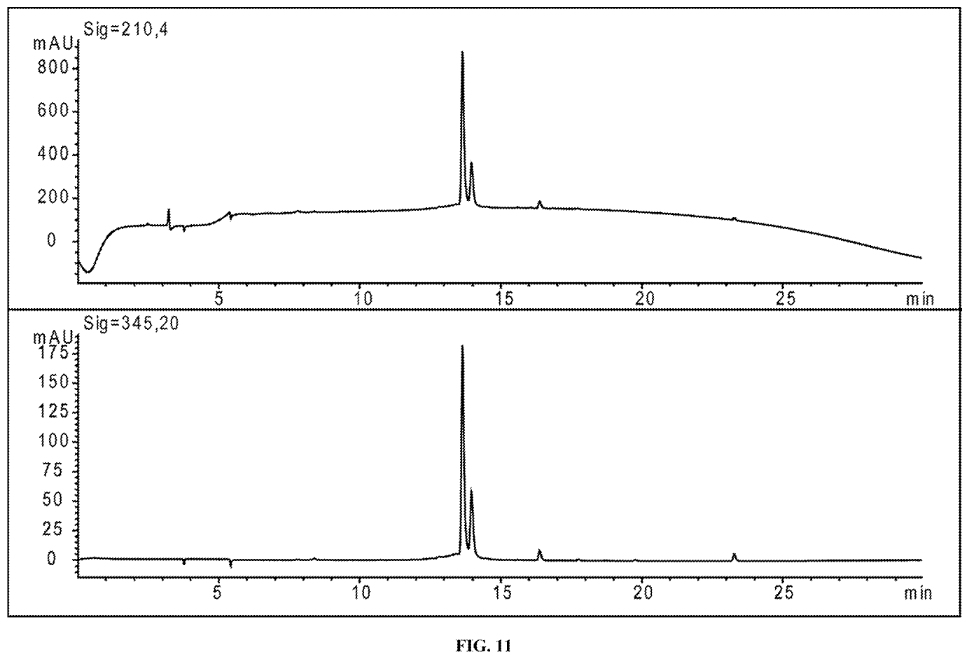

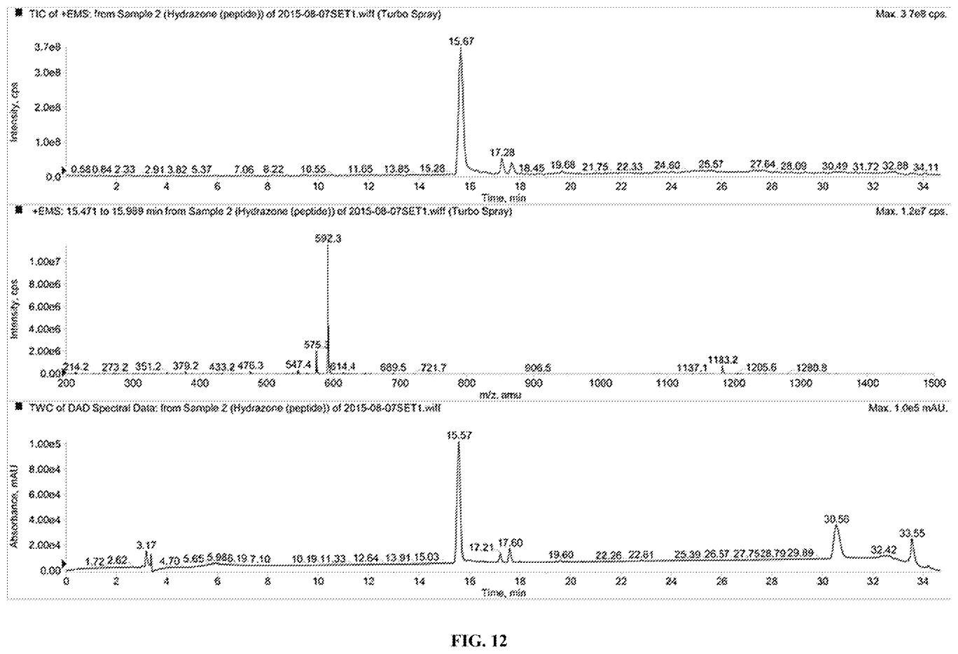

Product was removed from resin by treating the resin with 95% TFA in water solution. Cleavage from resin is carried out for one hour. TFA is collected in an RBF. Resin may be washed with extra TFA cleavage solution to extract all peptide into the solution. All TFA solutions are pooled and evaporated to dryness. Dry material is thoroughly washed with cold ether and the hydrazone was purified on a C18 column on a HPLC (peak identified by characteristic absorbance at 345 nm). Hydrazone was dried on rotovap and immediately dissolved in DMSO for diazo reaction. Compound may be characterized by MS for its exact mass. FIG. 11 shows the MS data confirming synthesis of the pure isolated hydrazone intermediate. FIG. 12 shows the following fragments were detected by mass spectrometer:

592.3 amu=M+1, Molecular ion peak

575.3 amu=M−16 peak, acylium ion peak

1183.2 amu=M+M+1 peak=dimer mass

476.3 amu=115 peak=loss of valine

614.3 & 547.4 amu—unknown

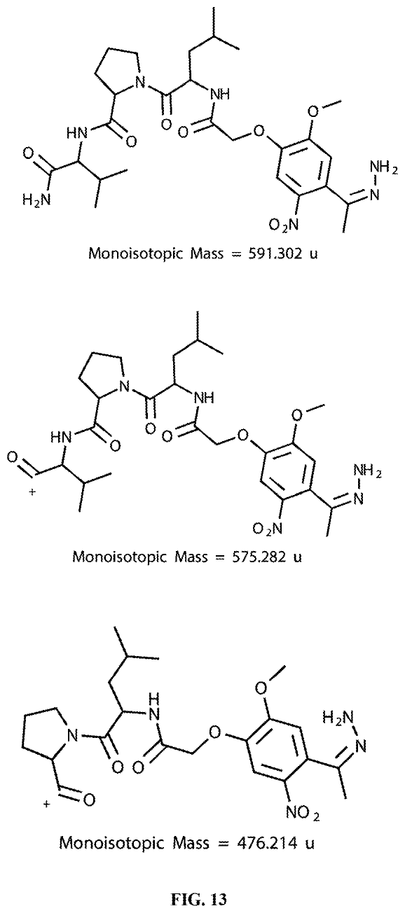

Fragments at detected at the exact mass of 591.30, 575.28 and 476.21 are depicted in FIG. 13.

Azine Side Reaction

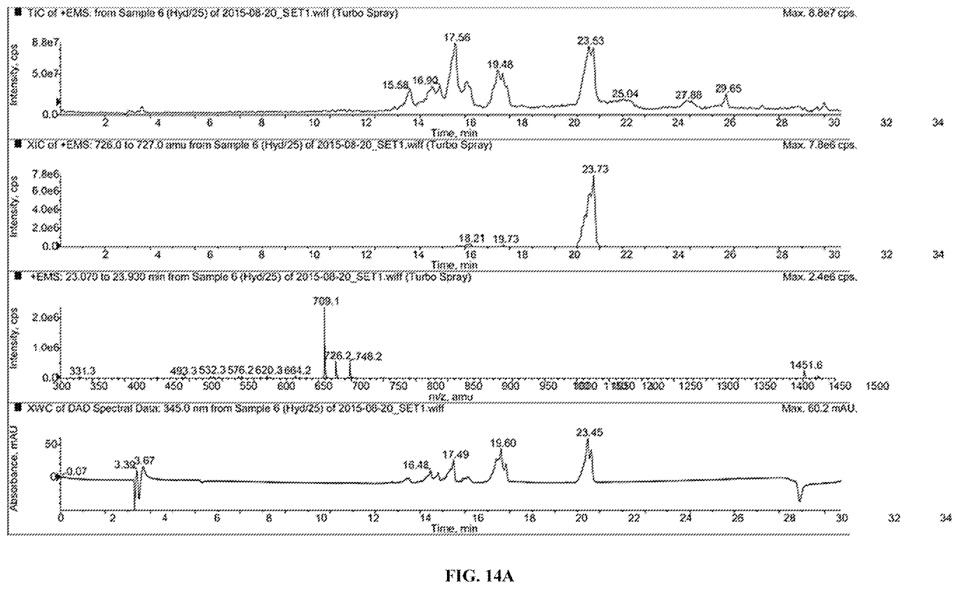

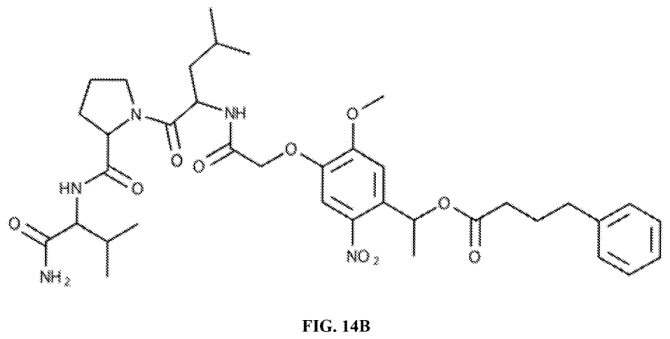

As discussed above, it was determined a side reaction forming an azine was occurring. Low hydrazone conditions were used to drive diazo production. FIG. 14 confirms diazotization at X/25 hydrazone with a model compound called PBA, showing that the adduct can be formed with less unproductive azine formation. These results with the model compound PBA indicated the conditions could be used with insulin, which was later demonstrated. Also, use of the unstable hydrazone immediately after synthesis significantly improves yields.

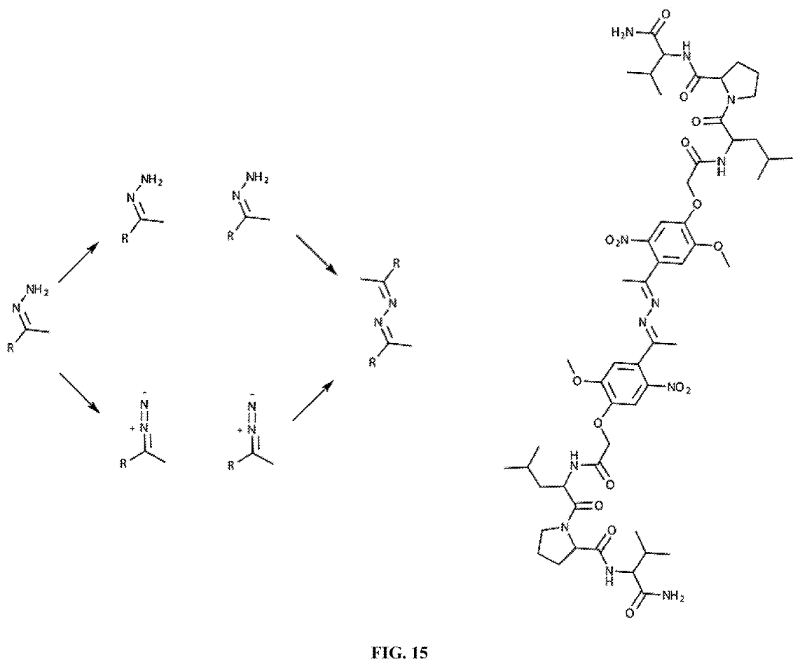

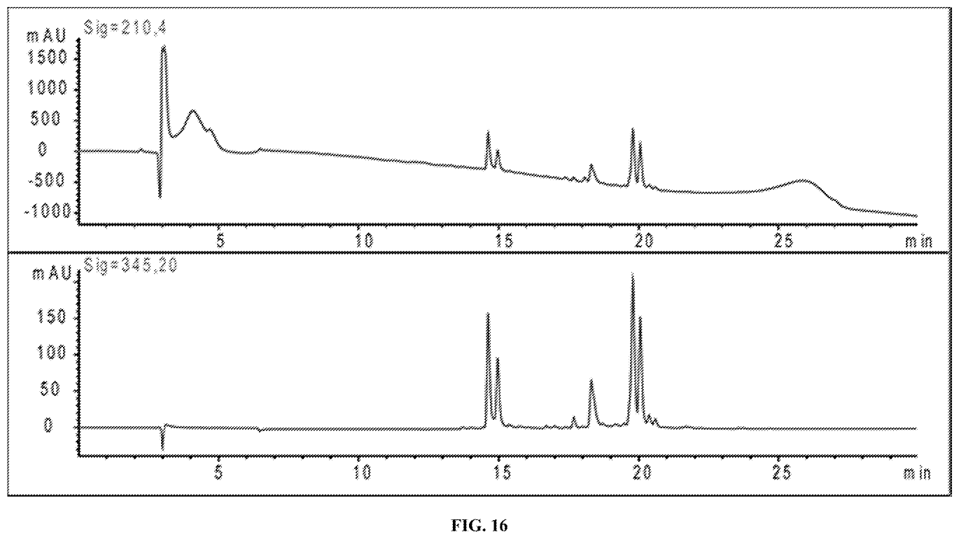

FIG. 15 depicts hurdles to the diazotization in which the azine is spontaneously formed during storage, which inhibits the diazotization reaction. FIG. 16 shows HPLC confirmation that hydrazone stored in a −20 freezer form azine. Based on AUC, 60% of the hydrazone is converted to azine.

Certain studies of the azine side reaction are summarized in Table 2. The hypothesis was that peptides associate, bringing hydrazones closer, leading to an increased rate of azine formation. The following experiments were carried out (MnO₂ diazotization). Standard conditions: 8.28 μmoles hydrazone (165.6 mM), 230 μmoles MnO₂ (20 mg), 45 minutes.

TABLE 2

| Reaction | Yield (of caged 4 PBA) | Optimum value |
|---|---|---|
| Increasing [hydrazone] | ↓ | . . . |
| Reducing [hydrazone] | ↑ | X/20 (8.28 mM), X/25 |
| Reducing time | ↑ | 30 min |
| Increasing shaker RPM | ↑ | 1500 |
| Reducing temperature | No significant difference | . . . |
| Changing solvent | No significant difference | . . . |

Val-Pro-Ile-Diazo Synthesis

Purified dried hydrazone was dissolved in least amount of DMSO and immediately quantitated using UV spectroscopy with 4470 M⁻¹ cm⁻¹ extinction coefficient. Quantitation should be done very quickly since the hydrazone is unstable and may form azines. Final concentration of the solution was adjusted to 12.2 mM (2.44 μmol) with anhydrous DMSO.

This solution (2.44 μmol) was immediately added to 117.8 mg of MnO2 in an Eppendorf tube. Reaction mixture was shaken vigorously for 45 minutes. Then the mixture was centrifuged at 12000 RPM for 5 minutes to remove MnO2. Diazo was collected in another Eppendorf tube and MnO2 may be washed with extra DMSO. Diazo was pooled and added to insulin solution immediately (described in next reaction step).

Val-Pro-Ile-Insulin Synthesis

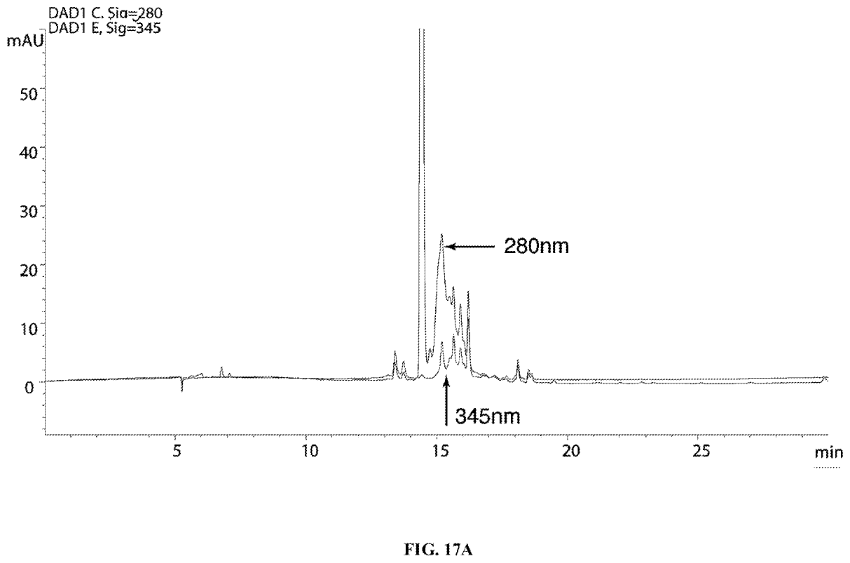

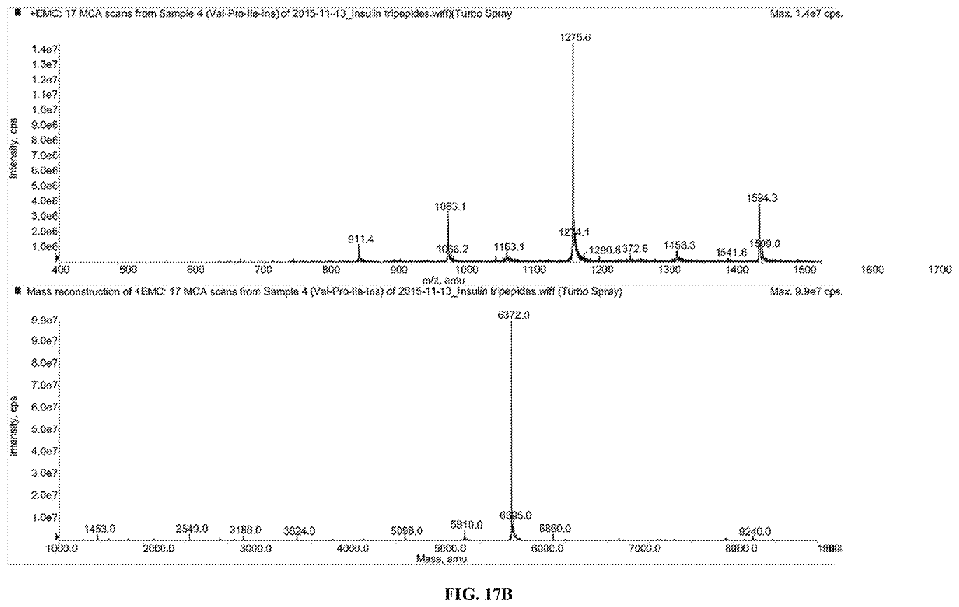

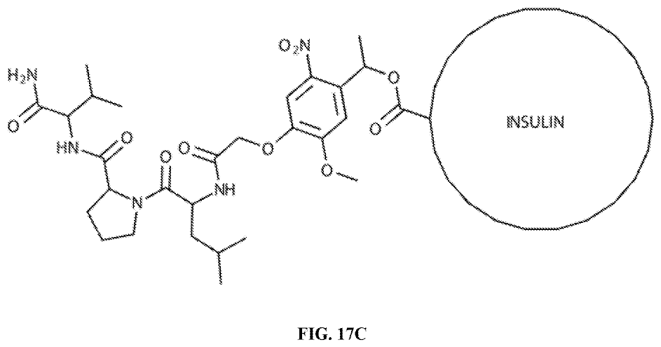

Insulin was weighed based on the diazo taken. Three times excess insulin was weighed (7.3 μmol, 42.5 mg). Diazo solution was directly added to dry insulin powder and allowed to dissolve and react for 24 hours. Val-Pro-Ile-Insulin was purified by C18 chromatography on HPLC, as shown in FIG. 17A. Material may be characterized by LCMS, as shown in FIG. 17B.

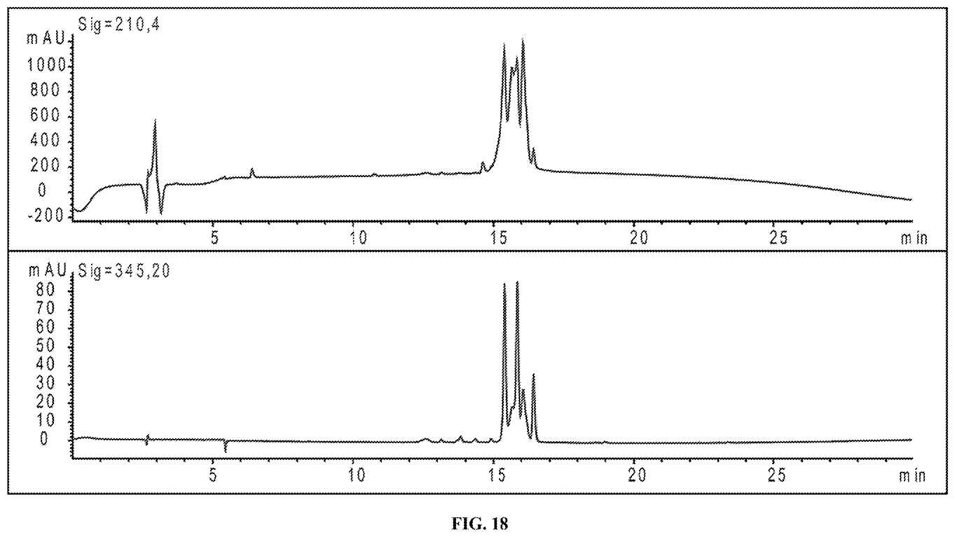

Solubility studies confirmed a significant drop (9×) in solubility of the drug conjugate vs. unmodified insulin, as shown if FIG. 18 and in Table 3, below.

TABLE 3

| Compound | Solubility (mM) | Solubility (mg/ml) |
|---|---|---|
| Val-Pro-Ile-insulin conjugate | 0.085 | 0.54 |
| Insulin | 0.708 | 4.11 |
| CD-insulin conjugate | 0.009 | 0.06 |

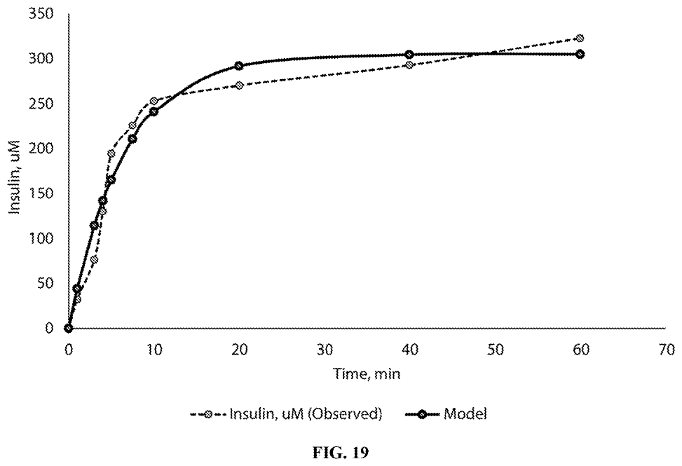

Release of native, unmodified insulin after photolysis is demonstrated in FIG. 19. (Equation B=A₀(1−exp(−kt)); A₀=305.1 uM, k=0.157 min⁻¹; CD-insulin, k=0.154 min⁻¹)

### Example 3. Synthesis of Drug Conjugate Comprising Insulin and Val-Val-Val Peptide

With the success attained with the conditions that worked with the Val-Pro-Ile moiety, drug conjugate comprising a Val-Val-Val-peptide was synthesized. Synthesis of a drug conjugate of the present invention comprising insulin as the drug molecule and valine-valine-valine as the non-polar moiety was carried out as follows:

Synthesis of Keto-Ester

As described above in Example 1.

Synthesis of NKA

As described above in Example 1.

Synthesis of Val-Val-Val-NKA

Val-Val-Val-NKA was made by solid phase synthesis.

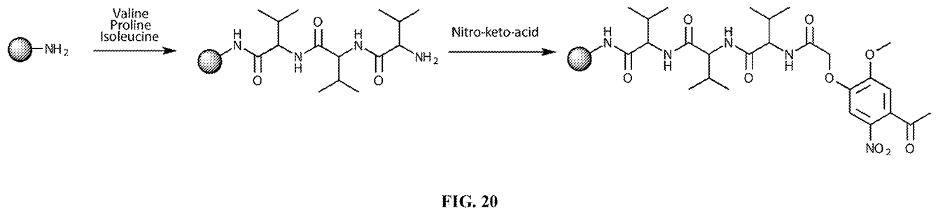

Synthesis is performed as described in the Val-Pro-Ile-Insulin section, except for the sequence of amino acids coupled varies here. An exemplary synthesis scheme is depicted in FIG. 20. The following amino acids were coupled in sequence:

Fmoc-Valine

Fmoc-Valine

Fmoc-Valine

NKA

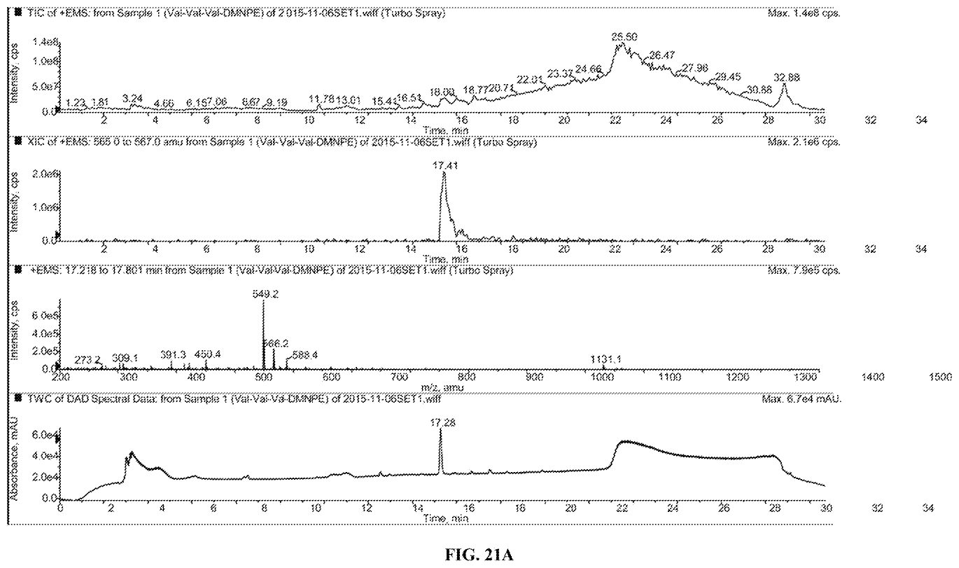

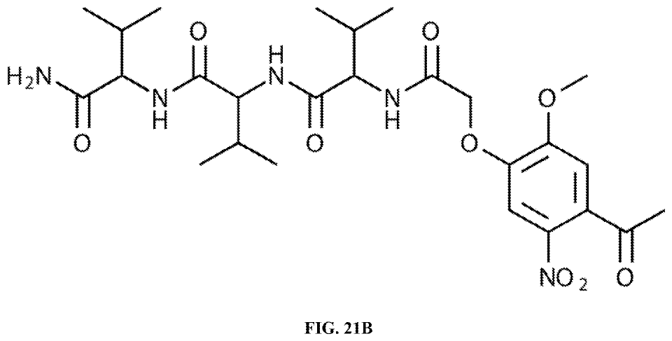

This resulted in desired compound and its mass may be characterized by MS, as shown in FIG. 21.

Val-Val-Val-Hydrazone Synthesis

Val-Val-Val-NKA (ketone) was converted to the hydrazone on resin. Resin was initially washed with a solvent mixture of NMP:ethanol (1:1).

Reaction was carried out in a sealed siliconized glass reaction vessel. 144.5 μmol of ketone (assuming 100% coupling on resin, 144.5 μmol of resin) was suspended in 5.4 ml of 1:1 NMP:ethanol solvent mixture. 216.75 μmol (130.8 μl) glacial acetic acid was added and mixed gently and thoroughly. 40 times excess hydrazine monohydrate (5780 μmol, 261.7 μl) was added to this mixture and reaction vessel was sealed tightly. This was shaken on an Eppendorf Thermomixer at 60 degrees C. Reaction was continued overnight. Resin was then thoroughly washed with NMP and DCM. Resin was allowed to dry completely.

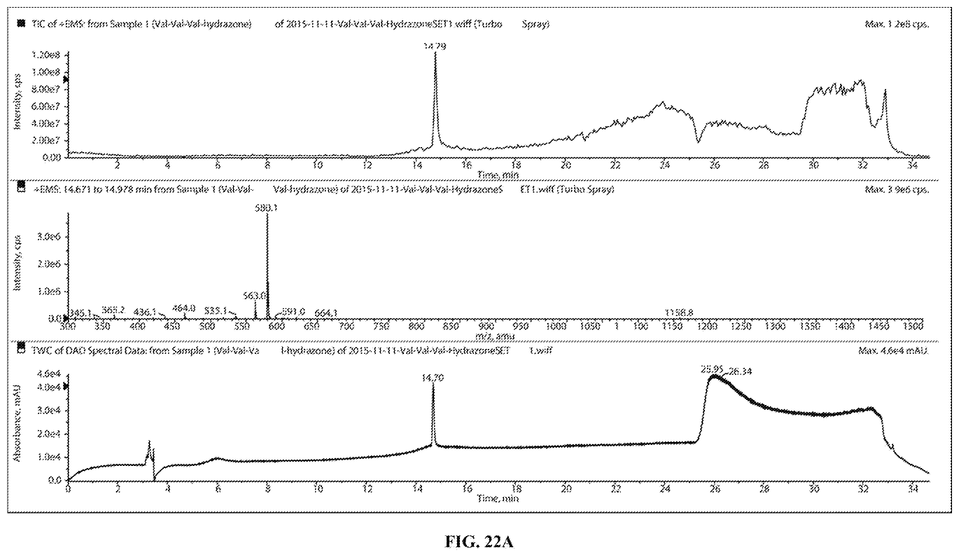

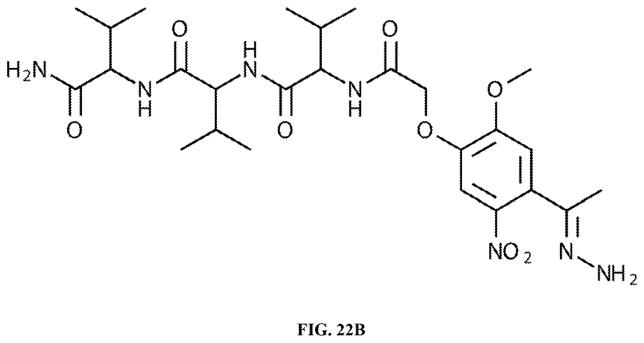

Product was removed from resin by treating the resin with 95% TFA in water solution. Cleavage from resin is carried out for one hour. TFA is collected in an RBF. Resin may be washed with extra TFA cleavage solution to extract all peptide into the solution. All TFA solutions are pooled and evaporated to dryness. Dry material is thoroughly washed with cold ether and the hydrazone was purified on a C18 column on a HPLC (peak identified by characteristic absorbance at 345 nm). Hydrazone was dried on rotovap and immediately dissolved in DMSO for diazo reaction. Compound may be characterized by MS for its exact mass, as shown in FIGS. 22 and 22A.

Val-Val-Val-Diazo Synthesis

Purified dried hydrazone was dissolved in least amount of DMSO and immediately quantitated using UV spectroscopy with 4470 M⁻¹ cm⁻¹ extinction coefficient. Quantitation should be done very quickly since the hydrazone is unstable and may form azines. Final concentration of the solution was adjusted to 1.84 mM (1.473 μmol) with anhydrous DMSO.

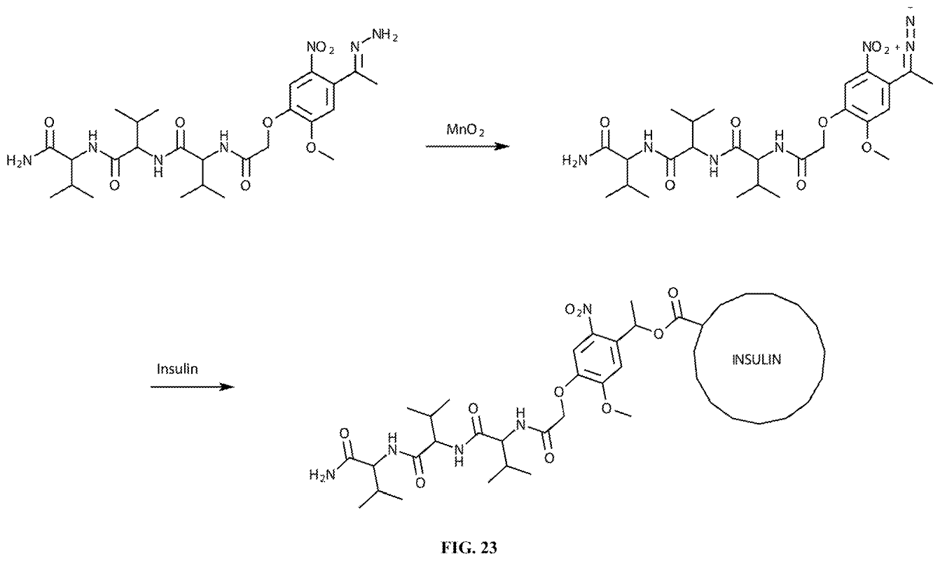

This solution (1.473 μmol) was immediately added to 71.2 mg of MnO2 in an Eppendorf tube. The reaction is shown in FIG. 23. Reaction mixture was shaken vigorously for 30 minutes. Then the mixture was centrifuged at 12000 RPM for 5 minutes to remove MnO2. Diazo was collected in another Eppendorf tube and MnO2 may be washed with extra DMSO. Diazo was pooled and added to insulin solution immediately (described in next reaction).

Val-Val-Val-Insulin Synthesis

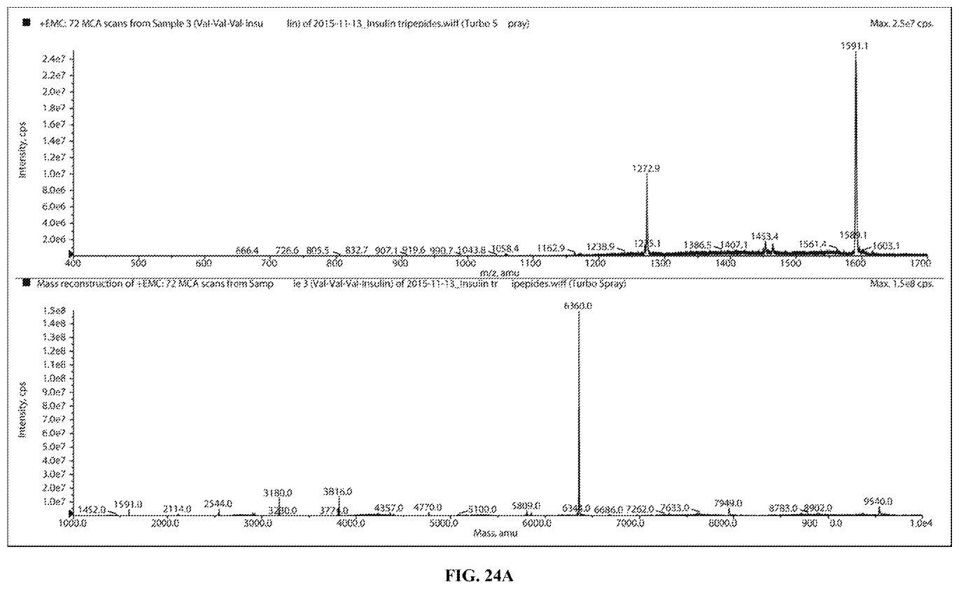

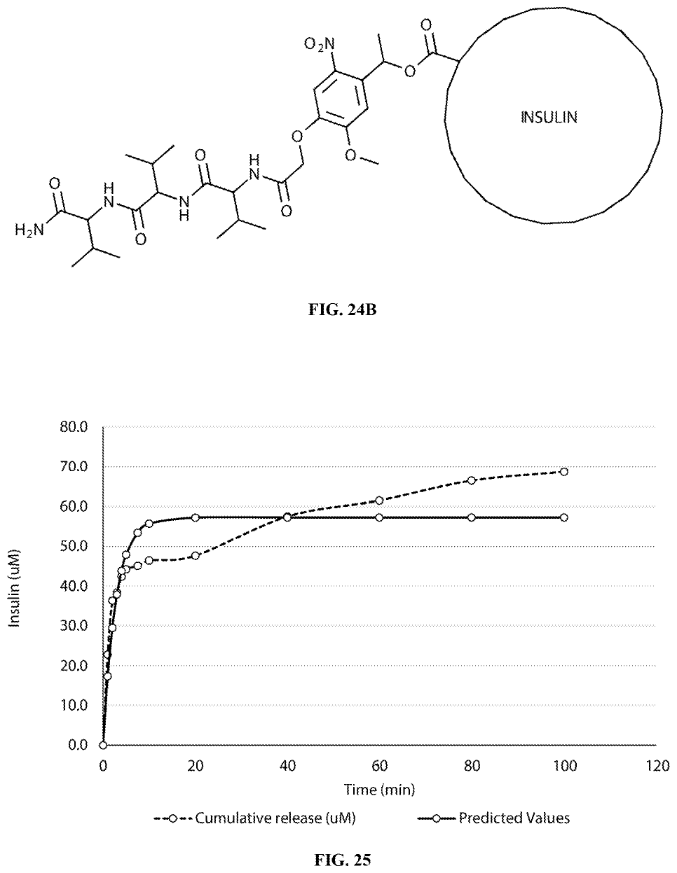

Insulin was weighed based on the diazo taken. Insulin was weighed in a 1:1 ratio with diazo (1.473 μmol, 8.56 mg) and dissolved in 1 ml DMSO. Diazo solution was added immediately to insulin solution and allowed to dissolve and react for 24 hours. The reaction is shown in FIG. 23. Val-Val-Val-Insulin was purified by C18 chromatography on HPLC. Material may be characterized by LCMS, as shown in FIG. 24. Release of native, unmodified insulin after photolysis is demonstrated in FIG. 25.

It was hypothesized solubility would be further lowered by the Val-Val-Val peptide being allowed to pack more efficiently than in the Val-Pro-Ile peptide. Solubility was determined as set forth in the following Table 4. The drug conjugate comprising the Val-Val-Val moiety demonstrated a 500 fold reduction in solubility from native insulin and was superior to other tested molecules. Solubility comparisons are set forth in Table 5 below.

TABLE 4

| | Amount in tube | Amount dissolved |
|---|---|---|
| Trial | (nmoles) | (nmoles) |
| 1 | 0.774 | 0.09 |
| 2 | 0.692 | 0.072 |
| 3 | 0.698 | 0.084 |

Excess VVV-Ins in Eppendorf → Shaken with 60 μl PBS (pH 7.2, 300 rpm, 10 min.) → centrifuged at 12000 rpm for 4 min → 50 μl supernatant analyzed

TABLE 5

| | Solubility | |
|---|---|---|
| | mg/ml | mM |
| Insulin | 4.11 | 0.708 |
| CDA-insulin | 0.06 | 0.009 |
| Val-Pro-Ile-insulin | 0.54 | 0.085 |
| Val-Val-Val-insulin | 0.009 | 0.0014 |

### Example 4. Synthesis of Drug Conjugate Comprising Insulin and Arg-Arg Peptide

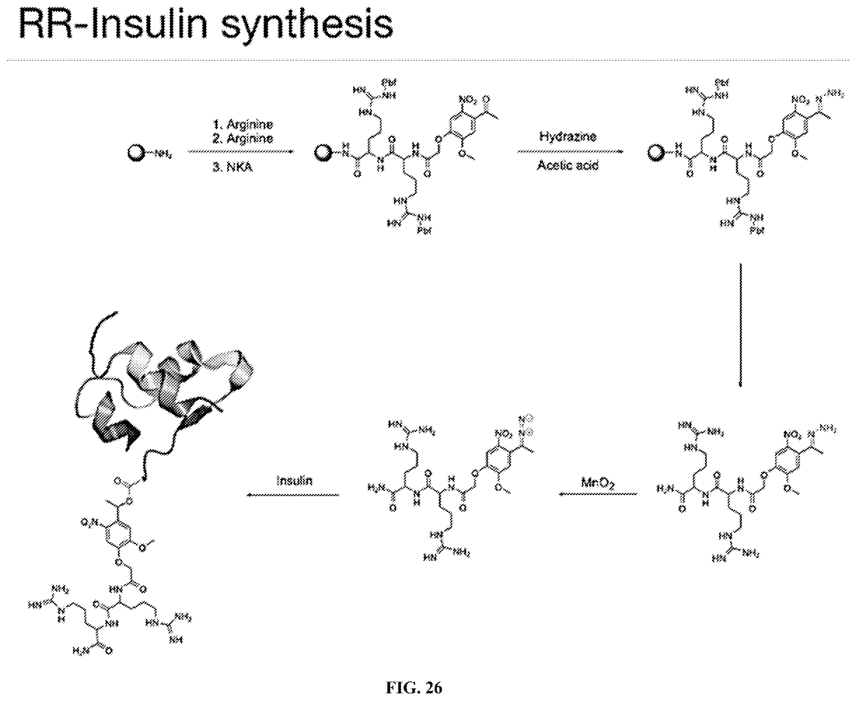

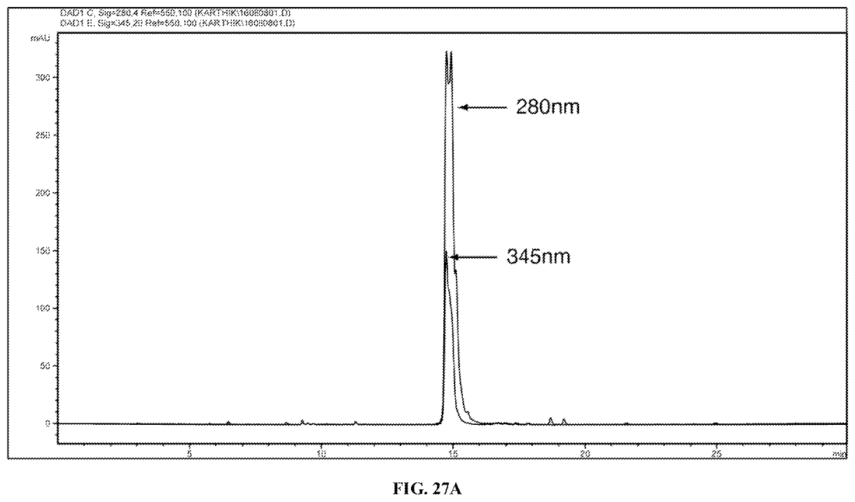

An exemplary synthetic scheme for a drug conjugate of the present invention comprising insulin as the drug molecule and an arginine-arginine peptide as the charged moiety is depicted in FIG. 26. The peptide is linked to insulin via a DMNPE based photocleavable group. The attachment point of DMNPE is a carboxyl group on the surface of the insulin. The modification of this carboxyl removes one negative charge (as the carboxyl can develop a negative charge at pH 7) and thus has the effect of adding an additional positive charge.

An exemplary synthesis was carried out as follows:

Synthesis of Keto-Ester

63.2 mmol (10.5 g) of acetovanillone, 69.2 mmol (13.5 g) tert-butylbromoacetate and 104 mmol (14.4 g) of potassium carbonate was added to 75 ml of dimethylformamide and stirred for 48 hours at room temperature. After 48 hours, water was added to the mixture until all salts were dissolved. This mixture was then partitioned between ethyl acetate and water. Pooled ethyl acetate fractions were washed with saturated NaCl, dried by addition of anhydrous magnesium sulfate. Ethyl acetate fraction was collected by filtration (to remove magnesium sulfate) and dried on a rotovap. This yields keto-ester as a white solid compound. Material was confirmed by MS and NMR.

Synthesis of NKA

3 ml of 70% nitric acid and 2 ml of acetic anhydride were cooled below 0 degrees C. Nitration mixture was first prepared by addition of 2 ml of acetic anhydride to 3 ml of 70% nitric acid on an ice bath to maintain the temperature below 0 degrees C.

3.6 mmol (1 g) of ketoester was dissolved in 3 ml (or slightly excess) of acetic anhydride. This solution was slowly added (dropwise) to the nitration mixture on ice bath. Care should be taken that the temperature should not rise. Reaction mixture was stirred for 2 hours on ice bath, and for another 4 hours at room temperature. A light-yellow precipitate may or may not be seen in the reaction mixture.

Reaction mixture was poured on to ice in a beaker and allowed to stand at 4 degrees overnight. Product can then be obtained by filtering the mixture and washing it extensively with cold water. Material may be recrystallized in MeOH/water mixture. Compound was characterized by MS and NMR.

Synthesis of Arg-Arg-NKA

Arg-Arg-NKA was made by solid phase synthesis.

CHEMMATRIX® Rink amide resin was used for this purpose. Dry resin (quantitated) was weighed and suspended in DCM, in a peptide synthesizer. DCM was removed by vacuum, and washed with excess of NMP five times.

Synthesis is carried out as shown in FIG. 9. Each wash step is performed by washing with NMP. Wash step is repeated 5 times to ensure that the resin is free from any reagents used in previous steps. Resin is finally suspended in a volume such that the total amines (on resin) in suspension is about 60 mM.

Amount of Fmoc-amino acid taken is 5 times excess of the amines present on resin, i.e. will have a concentration of 300 mM in solution. Fmoc-Amino acid is first activated before coupling it on to the resin. Activation is carried out by dissolving amino acid in NMP and adding a 1:1 molar equivalents of HATU and 1:2 molar equivalents of DIEA. This mixture is allowed to stand for about 10-15 minutes and immediately added to the Rink amide resin. Final conditions of the coupling is shown in Table 6 below. Coupling is carried out for 3 hours.

TABLE 6

| Reagent | Concentration |
|---|---|
| Amines (from Resin) | 60 mM |
| Fmoc-Amino acid | 300 mM |
| HATU | 300 mM |
| DIEA | 600 mM |

Resin is washed before capping free amines. Capping is performed using a solution of 10% acetic anhydride and 5% DIEA in NMP. This is performed for five minutes and the mixture is removed from resin by vacuum.

Resin is washed thoroughly (˜5 times) with NMP before removing the Fmoc group. Amines are deprotected by suspending the resin in NMP with 20% piperidine solution. This solution was removed after 5 minutes, and analysed on UV spec for characteristic fluorenyl group absorbance at 301 nm. Fresh 20% piperidine solution is again added to the resin and this process is repeated until 301 nm absorbance of the solution goes to 0 (or minimum), which is an indication that all the Fmoc groups have been removed in previous step.

Fmoc deprotection yields free amines and the next amino acid or carboxylic acid can be coupled after activation. This process is repeated with respective amino acid at each cycle until the desired peptide is obtained.

Product may be removed from a small amount of resin by treating it with 95% TFA, 5% water. TFA solution containing desired peptide is collected and washed with cold ether. Peptide is completely dried on rotovap and analysed by LCMS to confirm its mass.

Arg-Arg-NKA is synthesized per the procedure mentioned above, in the same sequence as:

1. Arginine

2. Arginine

3. Nitro-keto-acid

Material is stored on resin until used for next reaction.

Arg-Arg-Hydrazone Synthesis

Arg-Arg-NKA (ketone) was converted to the hydrazone on resin. Resin was initially washed with a solvent mixture of NMP:ethanol (1:1).

Reaction was carried out in a sealed siliconized glass reaction vessel. 235 mol of ketone (assuming 100% coupling on resin, 235 μmol of resin, 0.5 g) was suspended in 7 ml of 1:1 NMP:ethanol solvent mixture. 352.5 μmol (20.2 μl) glacial acetic acid was added and mixed gently and thoroughly. 40 times excess hydrazine monohydrate (9.4 mmol, 456.4 μl) was added to this mixture and reaction vessel was sealed tightly. This was shaken on an Eppendorf Thermomixer at 60 degrees C. Reaction was continued overnight. Resin was then thoroughly washed with NMP and DCM. Resin was allowed to dry completely.

Product was removed from resin by treating the resin with 95% TFA in water solution. Cleavage from resin is carried out for one hour. TFA is collected in an RBF. Resin may be washed with extra TFA cleavage solution to extract all peptide into the solution. All TFA solutions are pooled and evaporated to dryness. Dry material is thoroughly washed with cold ether. Crude hydrazone was dried on rotovap and immediately dissolved in DMSO for diazo reaction. Compound may be characterized by MS for its exact mass.

Note that the hydrazone is unstable and may self-react to form azines. Other by products were observed when hydrazone was left in solution for long time. It is recommended that the hydrazone is immediately converted to diazo and reacted with insulin (next reactions).

Arg-Arg-Diazo Synthesis

Dried hydrazone was dissolved in least amount of DMSO and immediately quantitated using UV spectroscopy with 4470 M⁻¹ cm⁻¹ extinction coefficient. Quantitation should be done very quickly since the hydrazone is unstable and may form azines and other unknown by products. Final concentration of the solution was adjusted to 11.04 mM (1.66 μmol) with anhydrous DMSO.

This solution (1.66 μmol) was immediately added to 690 μmol (60 mg) of MnO2 in an Eppendorf tube. Reaction mixture was shaken vigorously for 45 minutes. Then the mixture was centrifuged at 12000 RPM for 4 minutes to remove MnO2. Diazo was collected in another Eppendorf tube and MnO2 may be washed with extra DMSO. Diazo was pooled and added to insulin solution immediately (described in next reaction steps).

Arg-Arg-Insulin Synthesis

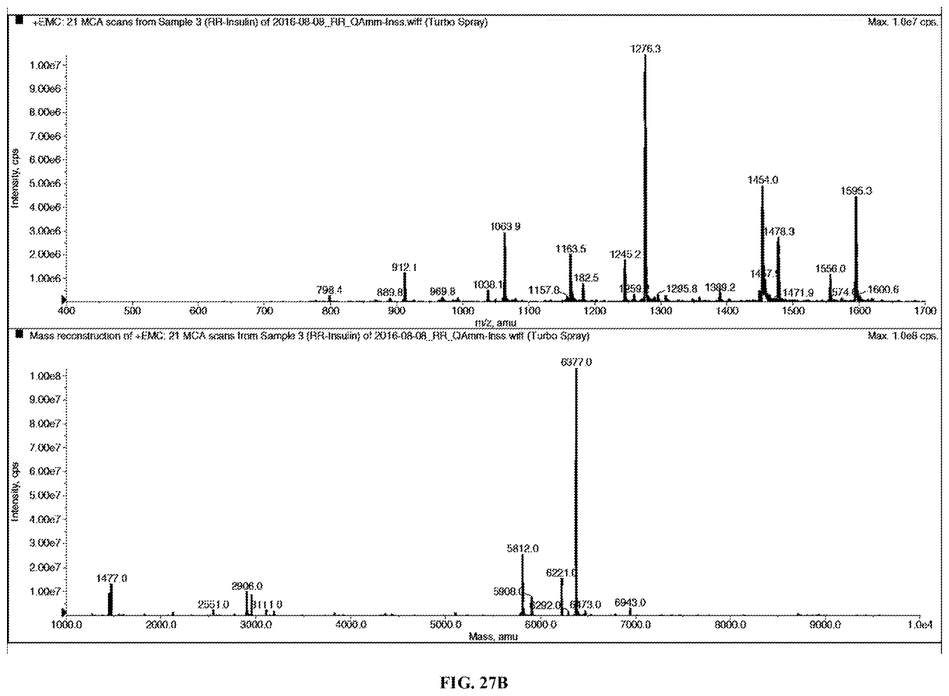

Insulin was weighed based on the diazo taken. Insulin was taken in a 1:1 ratio with assumed diazo molar quantity (1.66 μmol, 9.6 mg). Insulin was dissolved in 150 μl of DMSO. To this solution, diazo solution was added immediately after reaction and allowed react for 24 hours. Arg-Arg-Insulin was purified by C18 chromatography on HPLC with a shallow ACN gradient. A sharp gradient may not resolve the material since Insulin and Arg-Arg-Insulin have similar retention times. Material was characterized by MS, as shown in FIGS. 27A (insulin) and 28B (drug conjugate).

### Example 5. Synthesis of Drug Conjugate Comprising Insulin and G2PEA

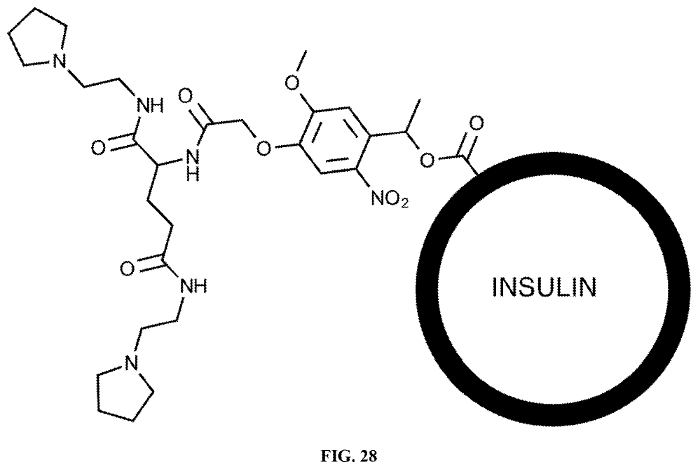

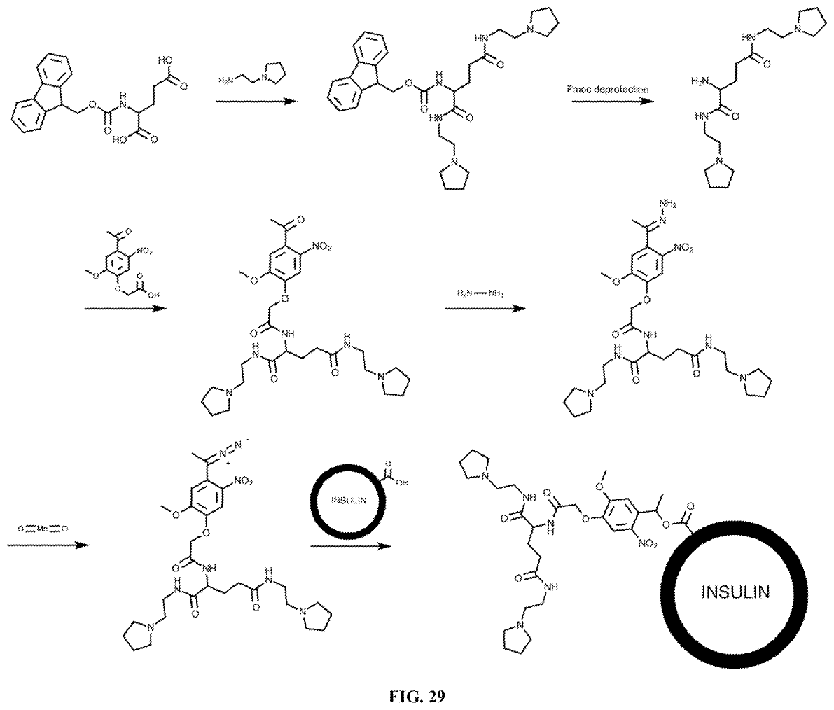

The structure of a drug conjugate of the present invention comprising insulin as the drug molecule and glutamic acid that has been condensed with two 1-(2-Aminoethyl)pyrrolidine moieties (G2PEA) as the non-polar moiety is depicted in FIG. 28.

In this example two positive charges were again added (through two basic amino groups on the charge tag) and one negative charge was taken away (through reaction of the tag with a carboxylic acid group on the target protein, blocking its negative charge). Data relating to this drug conjugate include structural proof (mass spectrometry), demonstration of modified isoelectric point (via gel analysis), and demonstration of differential solubility at two different pH values, ˜7 and ˜5. Two amino groups in the 5 membered rings confer positive charge because they are easily protonated. Attachment of the group to insulin blocks the negative charge of one of insulin's carboxylic acid groups.

An exemplary synthetic scheme is depicted in FIG. 29. An exemplary synthesis was carried out as follows:

Synthesis of Keto-Ester

As described above in Example 4.

Synthesis of Nitro-Keto-Acid (NKA)

As described above in Example 4.

Synthesis of Fmoc-G2PEA

269 μmoles Fmoc-glutamic acid (100 mg) was weighed in a reaction vial and dissolved in 4.5 ml of NMP. 805.5 μmol of HATU (306.3 mg) was added to solution and allowed to dissolve. Solution was allowed to stand for five minutes. DIEA (537 μmol, 93.54 μl) and pyrrolidinylethyleneamine (PEA; 805.5 μmol, 101.75 μl) were added to the solution and reacted overnight. Fmoc-G2PEA was purified on semi-preparative C18 column, dried on rotovap and was characterized by MS and NMR.

Amount of Fmoc-G2PEA purified was quantitated with UV-spectroscopy at 301 nm (Extinction coefficient=6800 M⁻¹ cm⁻¹ in ethanol)

Fmoc Deprotection and G2PEA Synthesis

Dried purified Fmoc-G2PEA was dissolved in 20 ml of acetonitrile and 20 ml of 40% dimethylamine solution was added. Solution was mixed thoroughly and allowed to stand for 30 minutes (RBF sealed as DMA is volatile). Solution was evaporated dryness.

Resulting dry material was dissolved in a solution of 20% DIEA in methanol. Material was allowed to dissolve completely and evaporated to dryness. This washing of material with 20% DIEA in methanol was repeated for about 3-5 times until all the DMA was evaporated from solution. Finally, the dry crude material was washed with cold ether multiple times and dried in vacuum.

Synthesis of NKA-G2PEA

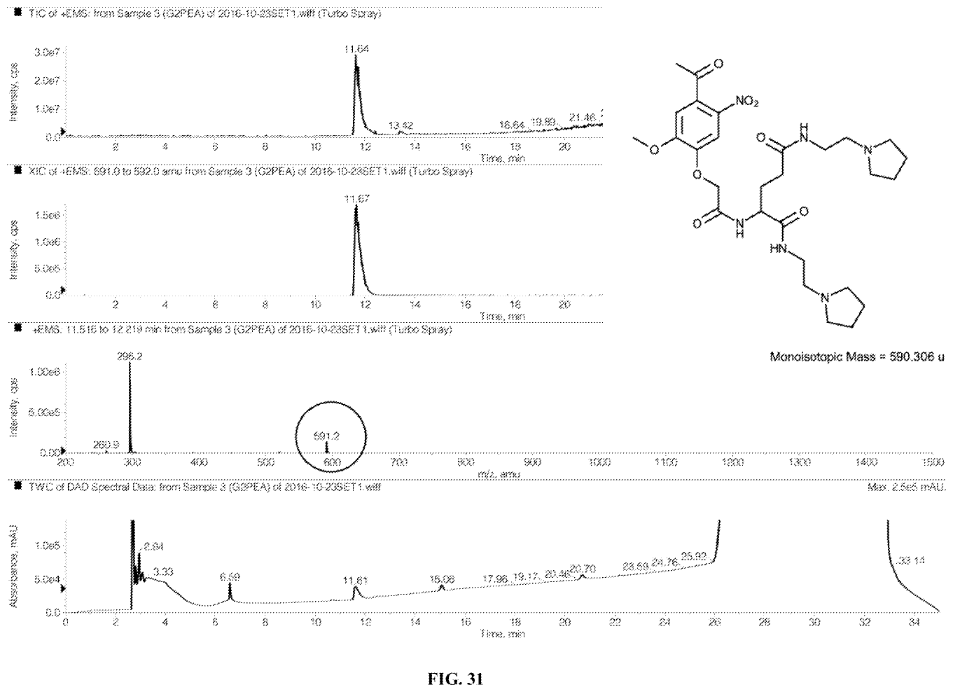

440 μmol (0.15 g) of G2PEA was dissolved in 5 ml NMP. To this solution, 660 μmol (0.18 g) NKA and 990 μmol (0.38 g) HATU were added and dissolved. 1.9 mmol (345 μl) of DIEA was added and reaction was allowed to proceed for at least 3 hours. G2PEA was purified on a semi-preparative column, dried and characterized by MS and NMR, as shown in FIG. 31.

Synthesis of G2PEA-Hydrazone

Purified G2PEA-NKA (ketone) was quantitated on UV spectroscopy at 345 nm with extinction coefficient of 4470 M⁻¹ cm⁻¹.

102.65 μmoles of ketone was dissolved in 6 ml of 1:1 Ethanol:ACN solution. To this, 15 μl of glacial acetic acid and 261.2 μl of hydrazine monohydrate were added and mixed thoroughly. Reaction was carried out in a sealed glass reaction vial at 90 degrees C. for 4 hours.

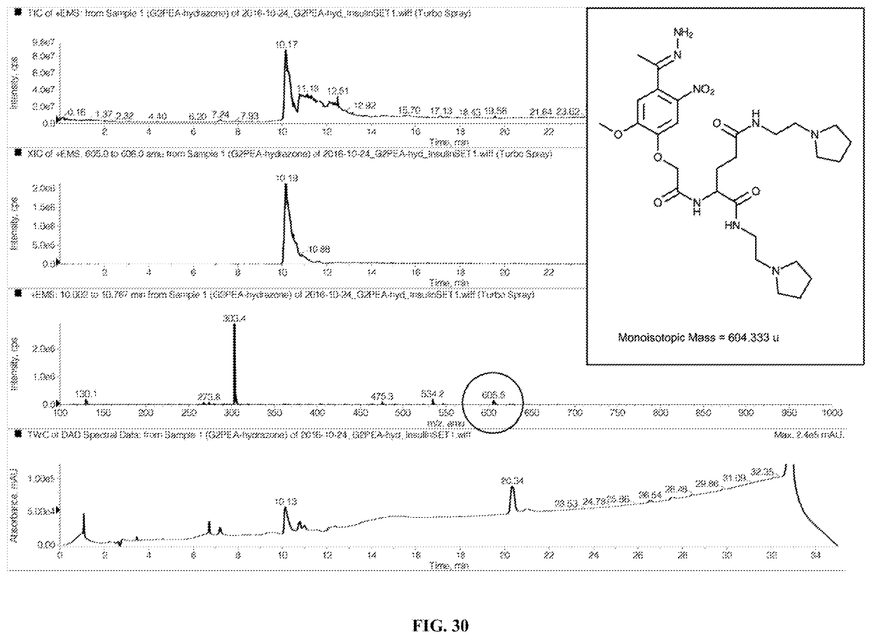

Reaction mixture was evaporated to dryness and washed with cold ether to remove any excess hydrazine and acetic acid. It is again dried in vacuum to remove ether completely. Presence of ether is not recommended as it reacts with MnO2 vigorously and results in a very low diazo yield. Characterization by LCMS is shown in FIG. 30. Note that the hydrazone is unstable and may self-react to form azines. Other by products were observed when hydrazone was left in solution for long time. It is recommended that the hydrazone is immediately converted to diazo and reacted with insulin (next reaction steps).

Synthesis of G2PEA-Diazo

Dried G2PEA-hydrazone was dissolved in least amount of anhydrous DMSO. The concentrated solution was quantitated using UV-spectroscopy at 345 nm using an extinction coefficient of 4470 M⁻¹ cm⁻¹. Final concentration was adjusted to 16.56 mM G2PEA-hydrazone by diluting the original solution with anhydrous DMSO.

132 μmol (16.56 mM) G2PEA-hydrazone was transferred into a glass reaction vial and 22.9 mmol (2000 mg) manganese dioxide was added and shaken vigorously for 45 minutes. Reaction mixture was immediately centrifuged at 12000 RPM for 5 min to remove manganese dioxide. Formation of diazo can be identified by a characteristic red color. Supernatant was collected into another Eppendorf tube. Manganese dioxide is washed with additional DMSO (as lower volume as possible) to recollect any trapped diazo. Solutions are pooled. Note that this diazo should be immediately reacted with Insulin as it is unstable. Compound characterized only by UV-Visible absorbance. Diazo will have a characteristic absorbance at 450 nm, unlike ketone and hydrazone that have absorbance only at 345 nm.

Synthesis of G2PEA-Insulin

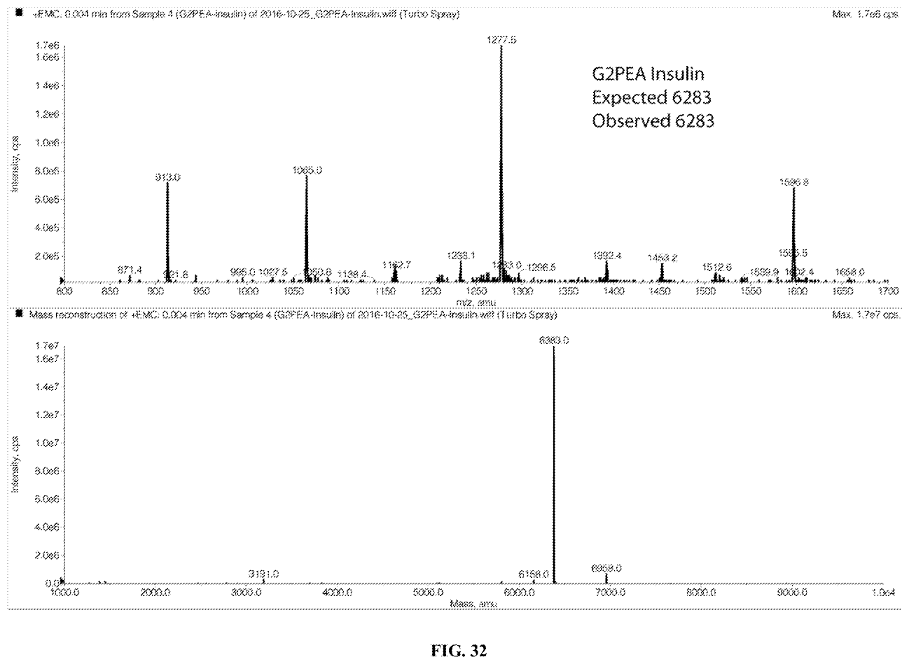

132 μmol (770 mg) of Insulin was weighed and dissolved in 8 ml anhydrous DMSO. Diazo solution (from previous reaction) was immediately added to the Insulin solution. Diazo was allowed to react for 24 hours. Reaction progress may be monitored on HPLC with a C18 column. G2PEA-Insulin resolves on C18 column on a very shallow acetonitrile gradient with its distinct retention time and may be purified as it elutes out of the column. Material can be identified with UV-spectroscopy and MS, as shown in FIG. 32.

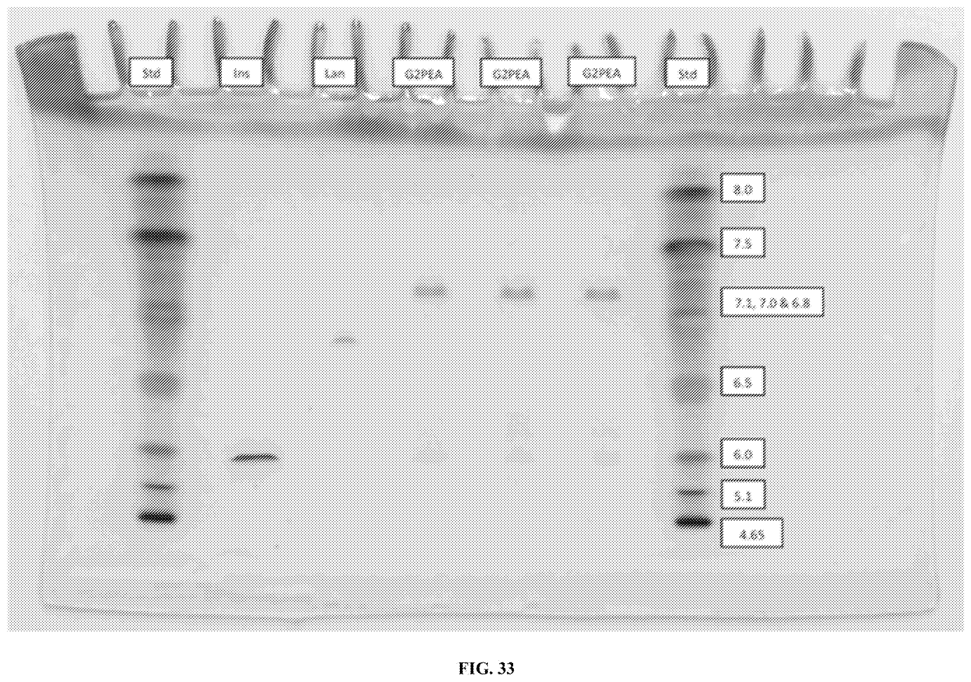

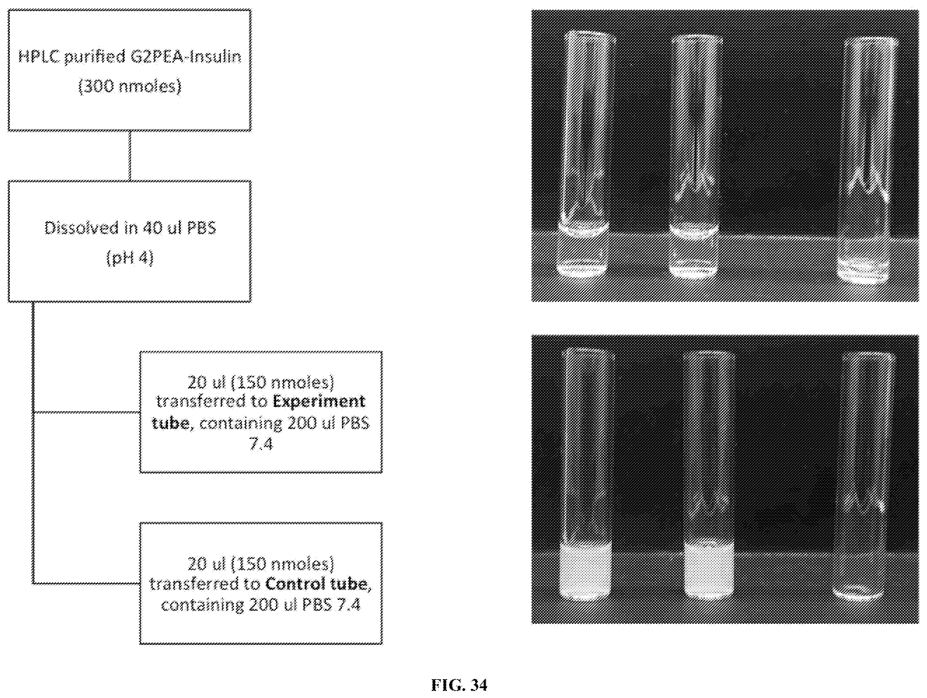

The altered isoelectric point of the drug conjugate is evidenced by the IEF gel shown in FIG. 33, which shows the new isoelectric point is ˜7.

In the experiment shown in FIG. 34, solubility of the drug conjugate at pH 4 (away from the pI) and at the pI (˜7) were compared. At the acidic pH 4, the solution is completely clear showing solubility. As soon as the pH is adjusted to ˜7 with buffer, the solution goes cloudy, as the material loses its solubility (due to being at or near the isoelectric point). In addition to this qualitative demonstration of G2PEA having reduced solubility at pH ˜7 and higher at lower pH, it was quantitatively determined that the solubility at pH7.2 was reduced by 75 times, as shown in Table 7.

TABLE 7

| | G2PEA-insulin drug | |
|---|---|---|
| | conjugate | Insulin |
| Solubility | 14.0 ± 1.3 μM | 1053.3 ± 158.1 μM |
| pH of buffer after | ~7.24 | ~7.3 |
| saturation | | |

Furthermore, the differential solubility at pH 5.4 (the pH of insulin) was determined, as shown in Table 8. The solubility of the G2PEA-insulin drug conjugate was 6.6 times higher than insulin solubility at pH 5.4.

TABLE 8

| | G2PEA-insulin drug | |
|---|---|---|
| | conjugate | Insulin |
| Solubility | 112.9 ± 17.8 μM | 17.1 ± 2.1 μM |
| pH of buffer after | ~5.39 | ~5.4 |
| saturation | | |

Combined, these results show that we successfully have altered the pI of the G2PEA insulin, and furthermore, that this has increased its solubility at low pH (5.4) and decreased it at the higher pH (˜7.3).

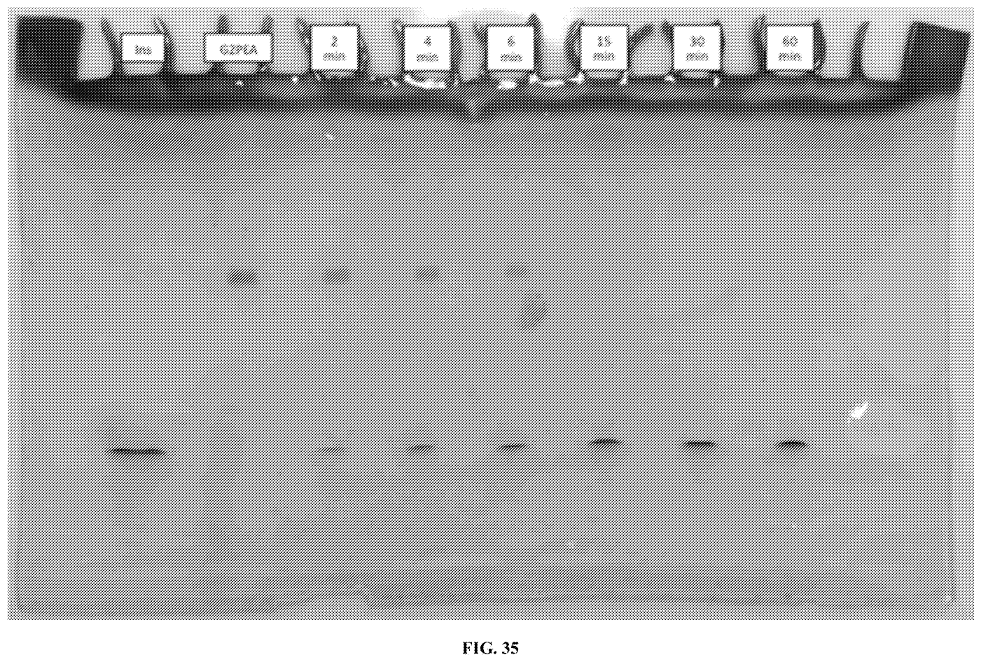

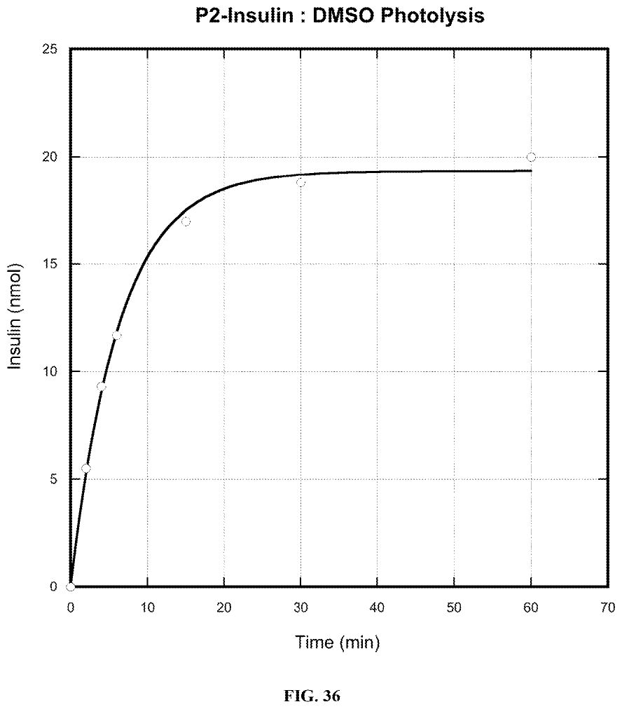

It was then shown that upon irradiation of the drug conjugate, insulin was released. The first demonstration, shown in FIG. 35, is with the drug conjugate dissolved in DMSO, and analyzed by gel. The upper band is the G2PEA insulin. The lower band is insulin. Over the irradiation time period (shown at the top of the gel) all of the G2PEA insulin is consumed, and insulin is released. This shows that the drug conjugate is capable of being converted with light to insulin. FIG. 36 shows the photolysis release profile.

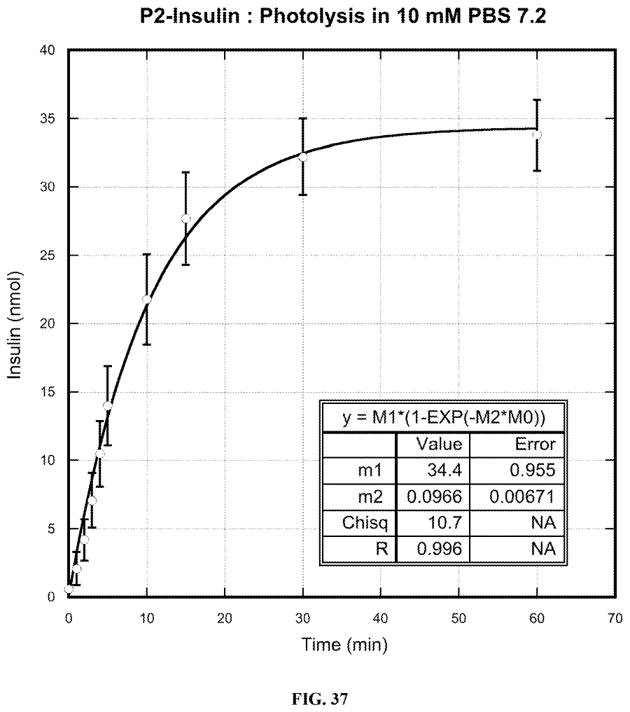

The drug conjugate was then examined in aqueous buffer (phosphate buffered saline or PBS) at pH 7.2. The material is largely insoluble at this pH, and so the release of insulin into the supernatant upon irradiation was observed, as shown in FIG. 37.

The preceding data demonstrates a new approach for creating photoactivated depots of therapeutics (typically proteins) using charged moieties, as described herein. This method uses the addition of charged groups to the protein via a photocleavable group or linker. The charges are selected to shift the isoelectric point, or pI, of the protein. This is the pH at which the protein will have no formal charge, and will also have its lowest solubility. Specifically, insulin was modified to make its modified structure have a pI near pH 7. This will allow it to be formulated at an acidic pH (eg 5) and be completely soluble. Upon injection, typically into the skin, it will precipitate out, forming a depot in the skin. This is because the skin is ˜7 pH. It was demonstrated that the synthesis of new reagents can modify insulin to shift its overall charge. It was demonstrated that the modified insulin has a pI close to 7. It was shown that this introduces the desired properties, high solubility at pH 5, low at pH 7. It was further shown that upon photolysis near pH 7, this insoluble material will release native, soluble insulin. These new materials therefore address some of the major problems associated with polymer-based photoactivated depot materials: low density of insulin on the material, and the use of polymers which need to be cleared from the system. The elimination of these problems increases the potential utility of the materials of the present invention.

From the foregoing, it will be seen that this invention is one well adapted to attain all ends and objectives herein above set forth, together with the other advantages which are obvious and which are inherent to the invention. Since many possible embodiments may be made of the invention without departing from the scope thereof, it is to be understood that all matters herein set forth or shown in the accompanying drawings are to be interpreted as illustrative, and not in a limiting sense. While specific embodiments have been shown and discussed, various modifications may of course be made, and the invention is not limited to the specific forms or arrangement of parts and steps described herein, except insofar as such limitations are included in the following claims. Further, it will be understood that certain features and subcombinations are of utility and may be employed without reference to other features and subcombinations. This is contemplated by and is within the scope of the claims.

We claim:

1. A composition for forming an implanted drug depot, said composition comprising a plurality of drug conjugates, wherein said drug conjugates comprise:
a solubility modulating portion comprising:
a biocompatible, bioresorbable moiety; and
a photocleavable group linked to said moiety; and
a drug molecule linked to said photocleavable group of said modulating portion;
wherein said drug conjugates are insoluble at physiological pH.

2. The composition of claim 1, wherein said moiety and modulating portion has a molecular weight of 2000 or less, preferably 1500 or less, more preferably 1000 or less.

3. The composition of claim 1, wherein said moiety is insoluble at physiological pH.

4. The composition of claim 1, wherein said moiety is non-polar.

5. The composition of claim 4, wherein said moiety is a peptide comprising 20 or fewer non-polar amino acids, preferably 15 or fewer, 10 or fewer, or 5 or fewer non-polar amino acids.

6. The composition of claim 5, wherein said moiety comprises 3 non-polar amino acids.

7. The composition of claim 1, wherein said moiety is comprised of amino acids selected from the group consisting of glycine, alanine, valine, leucine, isoleucine, proline, phenylalanine, methionine, tyrosine and tryptophan.

8. The composition of claim 7, wherein said moiety comprises a valine-proline-isoleucine peptide or a valine-valine-valine peptide.

9. The composition of claim 4, wherein said moiety is a substituted or unsubstituted hydrocarbon.

10. The composition of claim 9, wherein said moiety comprises cyclododecyl amine.

11. The composition any of claim 1, wherein said moiety has a charge that shifts the isoelectric point of the drug conjugate to a physiological pH.

12. The composition of claim 11, wherein said physiological pH is from 6.5 to 7.5.

13. The composition of claim 11, wherein said moiety comprises one or more groups selected from positive groups, negative groups and combinations thereof, wherein the combined charge of said moiety shifts the isoelectric point of the drug conjugate to a physiological pH.

14. The composition of claim 11, wherein said drug molecule is insulin and said moiety adds two positive charges to the drug conjugate.

15. The composition of claim 11, wherein said moiety comprises a peptide.

16. The composition of claim 16, wherein said peptide comprises amino acids selected from the group consisting of arginine, lysine and histidine.

17. The composition of claim 15, wherein said peptide comprises two amino acids.

18. The composition of claim 17, wherein said peptide is an arginine-arginine peptide.

19. The composition of claim 11, wherein said moiety comprises glutamic acid that has been condensed with two 1-(2-Aminoethyl)pyrrolidine moieties (G2PEA).

20. The composition of claim 1 wherein said drug is a therapeutic peptide.

21. The composition of claim 20 wherein said therapeutic peptide is insulin.

22. A method of administering a drug to a patient comprising:
implanting the composition of claim 1 into a patient to form said depot;
transdermally irradiating said implanted depot with light sufficient to cleave said photocleavable group and release said drug molecule from the drug conjugate;
wherein said released drug molecule is in its native form.

23. The method of claim 22 wherein said implanting step comprises injecting said depot cutaneously or subcutaneously.

24. A system for administering a drug to a patient comprising:
the composition comprising a drug conjugate according to claim 1; and
a light emitting device.

25. The system of claim 24 wherein said light emitting device is in the form of a band, patch, or bandage adapted to be positioned on said patient's skin.

26. The system of claim 24 wherein said light emitting device is programmed to provide light in response to a biological variable in a patient and wherein said system further comprises a sensor for measuring said biological variable to provide feedback to said light emitting device.

27. A composition for forming an implanted drug depot, said composition comprising a plurality of drug conjugates, wherein said drug conjugates comprise:
a solubility modulating portion comprising:
a biocompatible, bioresorbable moiety; and
a photocleavable group linked to said moiety; and
a drug molecule linked to said photocleavable group of said modulating portion;
wherein said drug conjugates are insoluble at physiological pH.

28. The composition of claim 27, wherein said moiety and modulating portion has a molecular weight of 2000 or less, preferably 1500 or less, more preferably 1000 or less.

29. The composition of claim 27 or 28, wherein said moiety is insoluble at physiological pH.

30. The composition of any of claims 27-29, wherein said moiety is non-polar.

31. The composition of claim 30, wherein said moiety is a peptide comprising 20 or fewer non-polar amino acids, preferably 15 or fewer, 10 or fewer, or 5 or fewer non-polar amino acids.

32. The composition of claim 31, wherein said moiety comprises 3 non-polar amino acids.

33. The composition of any of claims 27-32, wherein said moiety is comprised of amino acids selected from the group consisting of glycine, alanine, valine, leucine, isoleucine, proline, phenylalanine, methionine, tyrosine and tryptophan.

34. The composition of claim 33, wherein said moiety comprises a valine-proline-isoleucine peptide or a valine-valine-valine peptide.

35. The composition of claim 30, wherein said moiety is a substituted or unsubstituted hydrocarbon.

36. The composition of claim 35, wherein said moiety comprises cyclododecyl amine.

37. The composition any of claims 27-29, wherein said moiety has a charge that shifts the isoelectric point of the drug conjugate to a physiological pH.

38. The composition of claim 37, wherein said physiological pH is from 6.5 to 7.5.

39. The composition of claim 37 or 38, wherein said moiety comprises one or more groups selected from positive groups, negative groups and combinations thereof, wherein the combined charge of said moiety shifts the isoelectric point of the drug conjugate to a physiological pH.

40. The composition of any of claims 37-39, wherein said drug molecule is insulin and said moiety adds two positive charges to the drug conjugate.

41. The composition of any of claims 37-40, wherein said moiety comprises a peptide.

42. The composition of claim 41, wherein said peptide comprises amino acids selected from the group consisting of arginine, lysine and histidine.

43. The composition of claim 42 or 42, wherein said peptide comprises two amino acids.

44. The composition of claim 43, wherein said peptide is an arginine-arginine peptide.

45. The composition of any of claims 37-40, wherein said moiety comprises glutamic acid that has been condensed with two 1-(2-Aminoethyl)pyrrolidine moieties (G2PEA).

46. The composition of any of claims 27-45 wherein said drug is selected from the group consisting of ACE-inhibitors; anti-anginal drugs; anti-arrhythmias; anti-asthmatics; anti-cholesterolemics; anti-convulsants; anti-depressants; anti-diarrhea preparations; anti-histamines; anti-hypertensive drugs; anti-infectives; anti-inflammatory agents; anti-lipid agents; anti-manics; anti-nauseants; anti-stroke agents; anti-thyroid preparations; anti-tumor drugs; anti-tussives; anti-uricemic drugs; anti-viral agents; acne drugs; alkaloids; amino acid preparations; anabolic drugs; analgesics; anesthetics; angiogenesis inhibitors; antacids; anti-arthritics; antibiotics; anticoagulants; antiemetics; antiobesity drugs; antiparasitics; antipsychotics; antipyretics; antispasmodics; antithrombotic drugs; anxiolytic agents; appetite stimulants; appetite suppressants; beta blocking agents; bronchodilators; cardiovascular agents; cerebral dilators; chelating agents; cholecystokinin antagonists; chemotherapeutic agents; cognition activators; contraceptives; coronary dilators; cough suppressants; decongestants; deodorants; dermatological agents; diabetes agents; diuretics; emollients; enzymes; erythropoietic drugs; expectorants; fertility agents; fungicides; gastrointestinal agents; growth regulators; hormone replacement agents; hyperglycemic agents; hypnotics; hypoglycemic agents; laxatives; migraine treatments; mineral supplements; mucolytics; narcotics; neuroleptics; neuromuscular drugs; NSAIDS; nutritional additives; peripheral vasodilators; polypeptides; prostaglandins; psychotropics; renin inhibitors; respiratory stimulants; steroids; stimulants; sympatholytics; thyroid preparations; tranquilizers; uterine relaxants; vaginal preparations; vasoconstrictors; vasodilators; vertigo agents; vitamins; and wound healing agents.

47. The composition of claim 46, wherein the drug is a therapeutic peptide.

48. The composition of claim 47, wherein said therapeutic peptide is selected from the group consisting of insulin; glucagon; calcitonin; gastrin; parathyroid hormones; angiotensin; growth hormones; secretin; luteotropic hormones (prolactin); thyrotropic hormones; melanocyte-stimulating hormones; thyroid-stimulating hormones (thyrotropin); luteinizing-hormone-stimulating hormones; vasopressin; oxytocin; protirelin; peptide hormones such as corticotropin; growth-hormone-stimulating factor (somatostatin); G-CSG, erythropoietin; EGF; physiologically active proteins, such as interferons and interleukins; superoxide dismutase and derivatives thereof; enzymes such as urokinases and lysozymes; and analogues or derivatives thereof.

49. The composition of claim 48 wherein said therapeutic peptide is insulin.

50. A method of administering a drug to a patient comprising:
implanting the composition of any one of claims 27 to 49 into a patient to form said depot;
transdermally irradiating said implanted depot with light sufficient to cleave said photocleavable group and release said drug molecule from the drug conjugate;
wherein said released drug molecule is in its native form.

51. The method of claim 50 wherein said implanting step comprises injecting said depot cutaneously or subcutaneously.

52. A system for administering a drug to a patient comprising:
the composition comprising a drug conjugate according to any one of claims 27 to 49; and
a light emitting device.

53. The system of claim 52 wherein said light emitting device is in the form of a band, patch, or bandage adapted to be positioned on said patient's skin.

54. The system of claim 52 or 53 wherein said light emitting device is programmed to provide light in response to a biological variable in a patient and wherein said system further comprises a sensor for measuring said biological variable to provide feedback to said light emitting device.
